SEQUENCE LISTING (25 sequences)

SEQ ID NO: 1 - protein, 15 aa, Artificial
  Fragment of naturally occuring INGAP peptide
IGLHDPSHGTLPNGS

SEQ ID NO: 2 - protein, 15 aa, Artificial
  Fragment of naturally occuring INGAP Peptide Protein
IGLHDPTQGTEPNGE

SEQ ID NO: 3 - protein, 15 aa, Artificial
  Fragment of naturally occuring INGAP Peptide Protein
WIGLHDPTQGTEPNG

SEQ ID NO: 4 - protein, 14 aa, Artificial
  Fragment of naturally occuring INGAP Peptide Protein
IGLHDPTQGTEPNG

SEQ ID NO: 5 - protein, 15 aa, Artificial
  Modified fragment of Human REG3 Protein
IGLHDPTQGTEPNGE

SEQ ID NO: 6 - protein, 15 aa, Artificial
  Modified fragment of Human REG3 Protein
WIGLHDPTQGTEPNG

SEQ ID NO: 7 - protein, 14 aa, Artificial
  Modified fragment of Human REG3 Protein
IGLHDPTQGTEPNG

SEQ ID NO: 8 - protein, 16 aa, Artificial
  Modified fragment of Human REG3 Protein
IGLHDPTQGTEPNGEC

SEQ ID NO: 9 - protein, 16 aa, Artificial
  Modified fragment of Human REG3 Protein
WIGLHDPTQGTEPNGC

SEQ ID NO: 10 - protein, 15 aa, Artificial
  Modified fragment of Human REG3 Protein
IGLHDPTQGTEPNGC

SEQ ID NO: 11 - protein, 15 aa, Artificial
  Modified fragment of Human REG3 Protein
IGLHDPTQGTEPNGC

SEQ ID NO: 12 - protein, 17 aa, Artificial
  Modified fragment of Human REG3 Protein
WIGLHDPTQGTEPNGEC

SEQ ID NO: 13 - protein, 15 aa, Artificial
  Modified fragment of Human REG3 Protein
IGLHDPTQGTEPNGC

SEQ ID NO: 14 - protein, 16 aa, Artificial
  Modified fragment of Human REG3 Protein
IGLHDPTQGTEPNGEC

SEQ ID NO: 15 - protein, 16 aa, Artificial
  Modified fragment of Human REG3 Protein
WIGLHDPTQGTEPNGC

SEQ ID NO: 16 - protein, 15 aa, Artificial
  Modified fragment of Human REG3 Protein
IGLHDPTQGTEPNGC

SEQ ID NO: 17 - protein, 16 aa, Artificial
  Modified fragment of Human REG3 Protein
IGLHDPTQGTEPNGEC

SEQ ID NO: 18 - protein, 16 aa, Artificial
  Modified fragment of Human REG3 Protein
WIGLHDPTQGTEPNGC

SEQ ID NO: 19 - protein, 15 aa, Artificial
  Modified fragment of Human REG3 Protein
IGLHDPTQGTEPNGC

SEQ ID NO: 20 - protein, 16 aa, Artificial
  Modified fragment of Human REG3 Protein
IGLHDPTQGTEPNGEC

SEQ ID NO: 21 - protein, 16 aa, Artificial
  Modified fragment of Human REG3 Protein
WIGLHDPTQGTEPNGC

SEQ ID NO: 22 - protein, 15 aa, Artificial
  Modified fragment of Human REG3 Protein
IGLHDPTQGTEPNGC

SEQ ID NO: 23 - protein, 16 aa, Artificial
  Modified fragment of Human REG3 Protein
IGLHDPTQGTEPNGEC

SEQ ID NO: 24 - protein, 16 aa, Artificial
  Modified fragment of Human REG3 Protein
WIGLHDPTQGTEPNGC

SEQ ID NO: 25 - protein, 15 aa, Artificial
  Modified fragment of Human REG3 Protein
IGLHDPTQGTEPNGC

(19) United States
(12) Patent Application Publication
(10) Pub. No.: US 2009/0142338 A1
(43) Pub. Date: Jun. 4, 2009

(54) Methods and Compositions for Treating Type 1 and Type 2 Diabetes Mellitus and Related Conditions

(75) Inventor: Claresa S. Levetan, Rosemont, PA (US)

Correspondence Address: PEPPER HAMILTON LLP, ONE MELLON CENTER, 50TH FLOOR, 500 GRANT STREET, PITTSBURGH, PA, 15219, US

(73) Assignee: CUREDM, INC., Wynnewood, PA (US)

(21) Appl. No.: 11/852,000

(22) Filed: Sep. 7, 2007

Related U.S. Application Data
(63) Continuation-in-part of application No. 11/367,682, filed on Mar. 3, 2006.
(60) Provisional application No. 60/658,965, filed on Mar. 4, 2005.
(60) Provisional application No. 60/682,087, filed on May 18, 2005.
(60) Provisional application No. 60/684,819, filed on May 25, 2005.

(51) Int. Cl. A61K 39/395; A61P 3/10

(52) U.S. Cl. 424/133.1

(57) ABSTRACT

Embodiments of the present invention relate to compositions and methods of treating type 1 or type 2 diabetes mellitus or other conditions relating to metabolic dysfunction that may impact insulin secretion or action by administering an islet neogenesis agent in combination with an agent or agents that selectively inhibits, blocks or destroys the autoimmune destruction of pancreatic cells or agents that optimize function within existing islets in patients with type 1 diabetes, type 2 diabetes and related conditions.

### CROSS-REFERENCE TO RELATED APPLICATIONS

This application is a continuation-in-part of U.S. application Ser. No. 11/367,682 filed Mar. 3, 2006, which claims priority to U.S. Ser. No. 60/658,965, filed Mar. 4, 2005, U.S. Ser. No. 60/682,087, filed May 18, 2005 and U.S. Ser. No. 60/684,819, filed May 25, 2005, each of which, are incorporated herein by reference in their entireties.

### GOVERNMENT INTERESTS

Not applicable

### PARTIES TO A JOINT RESEARCH AGREEMENT

Not applicable

### INCORPORATION BY REFERENCE OF MATERIAL SUBMITTED ON A COMPACT DISC

Not applicable

### BACKGROUND

Not applicable

1. Field of Invention

Not applicable

2. Description of Related Art

Not applicable

### BRIEF SUMMARY OF THE INVENTION

Embodiments of the present invention provide methods for treating newly diagnosed or pre-existing type 1 diabetes mellitus in a patient comprising administering to said patient an agent that stimulates pancreatic islet cell regeneration and/or transformation of new insulin producing islets and administering one or more immune-tolerance agents. Embodiments of the present invention provide methods for treating newly diagnosed or pre-existing type 1 diabetes mellitus in a patient comprising administering to said patient an agent or agents that stimulates pancreatic islet cell regeneration and/or transformation of new insulin producing islets in combination with one or more immune tolerance agents. An agent that stimulates pancreatic islet cell regeneration, and/or transformation of new insulin producing islets includes, but is not limited to, Human proIslet Peptide (HIP), Optimized HIP, hamster INGAP and other islet neogenesis agents. Immune tolerance agents include, but are not limited to, mycophenolate mofetil, daclizumab, anti CD20 antibody (for example, rituximab), anti CD3 antibody including teplizumab (hOKT3 gamma 1 (Ala-Ala), also known as MGA031) and the monoclonal antibody TRX4 (ChAglyCD3), CTLA4-Ig (abatacept) a selective costimulation modulator as it inhibits the costimulation off cells, an anti-CD52 antibody, such as alemtuzumab (Campath-1H), a or humanized monoclonal antibody to T-cells, polyclonal anti-T-lymphocyte globulin (ATG), DiaPep277, anti-GAD antibody vaccine based on the 65 kDa isoform of the recombinant human glutamic acid decarboxylase protein (rhGAD65), and other approaches to immune suppression including, diazoxide. In certain embodiments, the method may further comprise administering Vitamin D or a derivative thereof, including, but not limited to Vitamin D3 (cholecalciferol) and 1,25 dihydroxy vitamin D. In certain embodiments, the method may further comprise administering a beta cell or islet function optimizing agent, which may improve beta cell or islet function within existing islets. Such agents include, but are not limited to, Glucagon Like Peptide-1 (GLP-1) and its analogs, Gastric Inhibitory Peptide/Glucose-Dependent Insulinoptropic polypeptide (GIP), Amylin, and its analog, Pramlintide, and GUM receptor agonists, such as Liraglutide (NN2211) and Exendin-4/exenatide, or compounds which halt the destruction of GLP-1, such as Dipeptidyl Peptidase-4 Inhibitors, (DPP-4 inhibitors), including but not limited to Vildagliptin, Sitagliptin, Saxagliptin, and PHX1149, gastrin, epidermal growth factor-1 and insulin sensitizing agents including the thiazolidinediones, including bin not limited to rosiglitazone and pioglitazone, AGI-1067, an anti-inflammatory antioxidant agent that works by inhibiting signaling pathways that are activated in response to oxidative stress and pro-inflammatory stimuli, Rimonabant and other drugs that block the cannabinoid receptor 1 (CB1), the gut peptide, peptide YY (PYY), inclusive of, but not limited to PYY3-36 (PYY) nasal spray, the hypothalamic neuropeptide Y (NPY) and drugs that impact the leptin, ghrelin, pro-opiomelanocortin/melanocortin pathways or the melanocortin receptor, orlistat, impacting gut absorption of fat, the centrally acting sibutramine, or acarbose, which delays carbohydrate absorption.

Embodiments of the present invention provide methods for treating newly diagnosed or preexisting type 2 diabetes in a patient, comprising administering to said patient an agent or agents that stimulates islet neogenesis and administering one or more beta cell or islet function optimizing agents, which may improve beta cell or islet function within exiting islets. Embodiments of the present invention provide methods for treating newly diagnosed or preexisting type 2 diabetes in a patient comprising administering to said patient an agent that stimulates islet neogenesis in combination with one or more beta cell or islet function optimizing agents. The one or more agents that stimulate islet neogenesis include HIP, Optimized HIP, hamster INGAP or other islet neogenesis agents capable of islet regeneration and/transformation, of new islets. Agents that may optimize beta cell or islet function within existing pancreatic islets include, but are not limited to, Glucagon Like Peptide-1 (GLP-1) and its analogs, Gastric Inhibitory Peptide/Glucose-Dependent Insulinoptropic polypeptide (GIP), Amylin, and its analog, Pramlintide, and GLP-1 receptor agonists, such as Liraglutide (NN2211) and Exendin-4/exenatide, or compounds which halt the destruction of GLP-1, such as Dipeptidyl Peptidase-4 Inhibitors, (DPP-4 inhibitors), including but not limited to Vildagliptin, Sitagliptin, Saxagliptin, and PHX1149. Other compounds which may improve existing islet function include: gastrin, epidermal growth factor-1 and insulin sensitizing agents including the biguanide, Metformin, and the thiazolidinediones, including but not limited to Rosiglitazone and Pioglitazone. Other agents that may impact pancreatic function that may be utilized with the islet cell, neogenesis agent include AGI-1067, an anti-inflammatory antioxidant agent that works by inhibiting signaling pathways that are activated in response to oxidative stress and pro-inflammatory stimuli, Rimonabant and other drugs that block the cannabinoid receptor 1 (CB1), gut peptide, PYY, inclusive of but not limited to PYY3-36 (PYY) nasal, spray, the hypothalamic neuropeptide Y (NPY) and drugs that impact the leptin, ghrelin, pro-opiomelanocortin/melanocortin pathways or the melanocortin receptor, orlistat, sibutramine and acarbose. In certain embodiments, the method may further comprise administering Vitamin D or a derivative thereof, including, but not limited to cholecalciferol and 1,25 dihydroxy vitamin D.

Embodiments of the present invention provide methods for treating pathologies in which there are metabolic impairments that may impact endocrine function that include but not limited to impairment in insulin secretion or action, including insulin resistance at the level of the adipose tissue, muscles or liver, including fasting hyperglycemia, insulin resistant syndrome, hyperglycemic conditions generally in children or adults and those with a family history of diabetes exhibiting an abnormal lasting glucose or insulin levels, metabolic syndrome, being overweight, obesity, polycystic ovarian syndrome (PCOS), anovulatory cycles, fasting hyperlipidemia/hypercholesterolemia, elevated lasting total cholesterol, elevated LDL and VLDL cholesterol, family history of diabetes and some forms of impotence and sexual dysfunction associated with such conditions, comprising administering to said patient an agent or agents that stimulates islet neogenesis in combination with one or more beta cell and islet function optimizing agents that may improve beta cell of islet function within existing islets. The one or more agents that stimulate islet neogenesis include HIP, Optimized HIP, hamster INGAP and/or other islet neogenesis agents capable of islet regeneration and/transformation of new islets. Agents that may optimize beta cell or islet function within, existing pancreatic islets include, but are not limited to, Glucagon Like Peptide-1 (GLP-1) and its analogs. Gastric Inhibitory Peptide/Glucose-Dependent Insulinoptropic polypeptide (GIP), Amylin, and its analog, Pramlintide, and GUM receptor agonists, such as Liraglutide (NN2211) and Exendin-4/exenatide, or compounds which halt the destruction of GUM, such as Dipeptidyl Peptidase-4 Inhibitors, (DPP-4 inhibitors), including but not limited to Vildagliptin, Sitagliptin, Saxagliptin, and PHX1149, gastrin, epidermal growth factor-1 and insulin sensitizing agents including the biguanide, Metformin, and the thiazolidinediones, including but not limited to Rosiglitazone and Pioglitazone, AGI-1067, an antiinflammatory antioxidant agent that works by inhibiting signaling pathways that are activated in response to oxidative stress and pro-inflammatory stimuli, Rimonabant and other drugs that block the cannabinoid receptor 1 (CB1), gut peptide, PYY, inclusive of, but not limited to PYY3-36 (PYY) nasal spray, the hypothalamic neuropeptide Y (NPY) and drugs that impact, the leptin, ghrelin, pro-opiomelanocortin/melanocortin pathways or the melanocortin receptor, orlistat, sibutramine or acarbose. In certain embodiments, the method may further comprise administering Vitamin D or a derivative thereof, including, but not limited to Vitamin D3 (cholecalciferol) and 1,25-dihydroxy vitamin D.

Embodiments of the present invention also provide kits comprising an agent that stimulates islet neogenesis in combination with one or more immune tolerance agents. Further embodiments provide kits further including Vitamin D or a derivative thereof. Further embodiments provide kits further including beta cell or islet function optimizing agents.

Embodiments of the present invention also provide kits comprising an agent that stimulates islet neogenesis in combination with one or more beta cell or islet function optimizing agents. Further embodiments provide kits further including Vitamin D or a derivative thereof.

Embodiments of the present invention provide a therapeutic composition comprising an agent that stimulates islet neogenesis and an immune tolerance agents.

Embodiments of the present invention provide a therapeutic composition comprising an agent that stimulates islet neogenesis and a beta cell or islet function optimizing agent.

### DESCRIPTION OF DRAWINGS

The file of this patent contains at least one photograph or drawing executed in color. Copies of this patent with color drawing(s) or photograph(s) will be provided by the Patent and Trademark Office upon request and payment of necessary fee.

For a fuller understanding of the nature and advantages of the present invention, reference should be had to the following detailed description taken in connection with the accompanying drawings, in which:

FIG. 1 is a graph depicting the insulin levels after incubation in culture with human pancreatic ductal tissue with HIP1, HIP2 and HIP3.

FIG. 2 is a graph depleting insulin levels in human pancreatic islet cultures after incubation with HIP1, HIP2, HIP3, hamster INGAP.

FIG. 3 is a graph depicting the insulin requirements in mice rendered diabetic with streptozotocin and treated with HIP1, HIP2, HIP3 and hamster INGAP.

FIG. 4 is a graph depicting the rate of fall in insulin requirements between placebo-treated and streptozotocin-treated mice rendered diabetic and HIP-treated mice, showing a significantly faster decline in insulin requirements among HIP-treated mice compared to control mice (p=0.004).

FIG. 5 depicts the increased islet mass and islet numbers identified by histological evaluation and insulin, staining. HIP2 and HIP3 had significantly greater total islet mass (p=0.05) and significantly increased total islet numbers (p=0.022).

FIG. 6 is an immunofluorescent stain for insulin on mouse pancreatic tissue treated with HIP.

FIG. 7A is a Western Blot analysis demonstrating human insulin expression from PANC-1 cells under non-reducing and reducing conditions in response to incubation with various HIP and Optimized HIPs. FIG. 7B are Ponceau Stains under non-reducing and reducing conditions in response to incubation with various HIP and Optimized HIPs.

FIG. 8A demonstrates PANC-1 cells treated with HIP2, and Optimized HIP peptides for four days, with pictures taken on day 7 at 200× magnification. FIG. 8B demonstrates the progression of PANC-1 cell morphology changes through 7 days (control, HIP2 and HIP2B), with pictures taken on days 1, 2, 3, 5 and 7 at 200× magnification. FIG. 8C demonstrates progression morphological changes of PANC-1 cells treated with control and Optimized HIPs (HIP2 Dimer and HIP2 PEG).

FIG. 9 is a stain for CK19 and DAP1 to show nuclei and insulin in human pancreatic cells following administration of HIP2B.

FIG. 10 is graph depicting glucose levels of three NOD mice after treatment with placebo and lysophylline (LSF), HIP 2 and LSF, and HIP2B and LSF.

FIG. 11 depicts the translocation of the HIP receptor after stimulation with HIP and Optimized HIP. A Cy3 double antibody immunohistochemical staining of PANC-1 cells was performed after treatment with 150 μM HIP and Optimized HIP peptides for 48 hours, demonstrating the translocation of the HIP receptor from the cell membrane of PANC-1 cells to the cytoplasm upon stimulation with HIP and Optimized HIP.

FIG. 12 demonstrates exposure adjusted PANC-1 cells in SFM and TSFM with HIP and Optimized HIP2B peptides.

### DETAILED DESCRIPTION

Before the present compositions and methods are described, it is to be understood that this invention is not limited to the particular processes, compositions, or methodologies described, as these may vary. It is also to be understood that the terminology used in the description is for the purpose of describing the particular versions or embodiments only, and is not intended to limit the scope of the present invention which will be limited only by the appended claims. Unless defined, otherwise, all technical and scientific terms used herein have the same meanings as commonly understood by one of ordinary skill in the art. Although any methods and materials similar or equivalent to those described herein can be used in the practice or testing of embodiments of the present invention, the preferred methods, devices, and materials are now described. All publications mentioned herein, are incorporated by reference in their entirety. Nothing herein is to be construed as an admission that the invention is not entitled to antedate such disclosure by virtue of prior invention.

The following definitions are provided to assist the reader. Unless otherwise defined, all terms of art, notations and other scientific or medical terms or terminology used herein are intended to have the meanings commonly understood by those of skill in the chemical and medical arts. In some cases, terms with commonly understood meanings are defined herein for clarity and/or for ready reference, and the inclusion of such definitions herein should not necessarily be construed to represent a substantial difference over the definition of the term as generally understood in the art.

It must also be noted that as used herein and in the appended claims, the singular forms “a”, “an”, and “the” include plural reference unless the context clearly dictates otherwise. Thus, for example, reference to a “fibroblast” is a reference to one or more fibroblasts and equivalents thereof known to those skilled in the art, and so forth.

As used herein, the term “about” means plus or minus 10% of the numerical value of the number with which it is being used. Therefore, about 50% means in the range of 45%-55%.

As used herein, “HIP” refers to a Human proIslet Peptide, preferably HIP1, HIP2, or HIP3.

As used herein, “Optimized HIP” refers to variations of HIP, HIP1 and/or HIP2 wherein the peptide has been modified to increase the stability, solubility or bioavailability of HIP, HIP1 or HIP2 as described in the invention. Stability refers to the peptide's resistance to degradation by in-serum proteases which target and degrade non-Optimized HIP3, HIP1 and/or HIP2. Bioavailability refers to the amount of peptide available for in vivo therapeutic use by the target cells, pathways and/or systemic mechanisms based on the peptide's ability to avoid degradation by proteases and other systemic pathways that degraded non-Optimized HIPS, HIP1 and/or HIP2. Preferably, Optimized SOP refers to HIP3, HIP1 and/or HIP 2 that are blocked by the addition of an N-terminal amide group and a C-terminal acetyl group, pegylated, and a combination thereof.

As used herein, “treating” a condition or patient refers to taking steps to obtain beneficial or desired results, including clinical results. For purposes of this invention, beneficial or desired clinical results include, but are not limited to, alleviation or amelioration of one or more symptoms of diabetes, diminishment of extent of disease, delay or slowing of disease progression, amelioration, palliation or stabilization of the disease state, and other beneficial results described below. Symptoms of diabetes include low or inadequate levels of insulin or insulin activity, frequent urination, excessive thirst, extreme hunger, unusual weight loss, increased fatigue, irritability, blurry vision, genital itching, odd aches and pains, dry mouth, dry or itchy skin, impotence, vaginal yeast infections, poor healing of cuts and scrapes, excessive or unusual infections, hyperglycemia, loss of glycemic control, fluctuations in postprandial blood glucose, fluctuations in blood glucagon, fluctuations in blood triglycerides and include reduction in rate of or diminution of or improved outcomes with conditions that are accelerated by and/or occur because of or more frequently with earlier diabetes including microvascular and microvascular disease inclusive but limited cerebrovascular impairment with or without, stroke, angina, coronary heart disease, myocardial infarction, peripheral vascular disease, nephropathy, kidney impairment, increased proteinuria, retinopathy, neovascularization of vessels in the retina, neuropathy including central, autonomic and peripheral neuropathy that may lead to loss of sensation of extremities and amputation and/or from neuropathy or diminished vascular flow, skin conditions including but not limited to diabetic dermopathy, Necrobiosis Lipoidica. Diabeticorum, bullosis diabeticorum, scleroderma diabeticorum, granuloma annulare bacterial skin infections, but limited to Staphylococcus, which can result in deeper infections. Diabetes may be diagnosed by methods well known to one of ordinary skill in the art. For example, commonly, diabetics have a plasma blood glucose result of greater than 126 mg/dL of glucose. Prediabetes, which may also be treated by the compositions and methods of the invention, is commonly diagnosed in patients with a blood glucose level between 100 and 125 mg/db of glucose. Other symptoms may also be used to diagnose diabetes, related diseases and conditions, and diseases and conditions affected by diminished pancreatic function.

As used herein, “reduction” of a symptom or symptoms (and grammatical equivalents of this phrase) means decreasing of the severity or frequency of the symptom(s), or elimination of the symptom(s).

The term “inhibiting” includes the administration of a compound of the present invention to prevent, the onset of the symptoms, alleviating, the symptoms, or eliminating the disease, condition or disorder.

As used herein, a “pathology associated with impaired pancreatic function” is one in which the pathology is associated with a diminished capacity in a subject for the pancreas of the subject to produce and/or secrete hormones and/or peptides. Preferably this hormone or cytokine is insulin. Pathologies that are associated with impaired pancreatic function include type 1 diabetes, new onset type 1 diabetes, type 2 diabetes, latent autoimmune diabetes of adulthood, pre-diabetes, impaired lasting glucose, impaired glucose tolerance, lasting hyperglycemia, insulin resistant syndrome, hyperglycemic conditions generally in children or adults and those with a family history of diabetes exhibiting an abnormal fasting glucose or insulin levels, metabolic syndrome, overweight, obesity, hyperlipidemia, cholesterolemia, hypertriglyceridemia, eating disorders, polycystic ovarian syndrome, anovulatory cycles, fasting hyperlipidemia/hypercholesterolemia, elevated fasting total cholesterol, elevated LDL and VLDL cholesterol, family history of diabetes and forms of impotence or sexual dysfunction associated with such conditions. “New onset” or “newly diagnosed” is defined as having been diagnosed with diabetes within the past 3 months, whereas “pre-existing” is defined as having been diagnosed with diabetes 3 months ago or longer.

As used herein, “administering” or “administration of” a drug or therapeutic to a subject (and grammatical equivalents of this phrase) includes both direct administration, including, self-administration, directly into or onto a target tissue or to administer a therapeutic to a subject whereby the therapeutic positively impacts the tissue to which it is targeted, and indirect administration, including the act of prescribing a drug. For example, as used herein, a physician who instructs a patient to self-administer a drug and/or provides a patient with a prescription for a drug is administering the drug to the patient.

As used herein, a “subject” or “patient” is a mammal, typically a human, but optionally a mammalian animal of veterinary importance, including but not limited to horses, cattle, sheep, dogs, and cats.

As used herein, a “manifestation” of a disease refers to a symptom, sign, anatomical state (e.g., lack of islet cells), physiological state (e.g., glucose level), or report (e.g., triglyceride level) characteristic of a subject with the disease.

As used herein, a “therapeutically effective amount” of a drug or agent is an amount of a drag or agent that, when administered to a subject with a disease or condition will have the intended therapeutic effect, e.g., alleviation, amelioration, palliation or elimination of one or more manifestations of the disease or condition in the subject. The full therapeutic effect does not necessarily occur by administration of one dose and may occur only after administration of a series of doses. Thus, a therapeutically effective amount may be administered in one or more administrations.

As used herein, a “prophylactically effective amount” of a drug is an amount of a drug that, when administered to a subject, will have the intended prophylactic effect, e.g., preventing or delaying the onset (or reoccurrence) of disease or symptoms, or reducing the likelihood of the onset (or reoccurrence) of disease or symptoms. The full prophylactic effect does not necessarily occur by administration of one dose and may occur only after administration of a series of doses. Thus, a prophylactically effective amount may be administered in one or more administrations.

By “pharmaceutically acceptable”, it is meant the carrier, diluent or excipient must be compatible with the other ingredients of the formulation and not deleterious to the recipient thereof.

As used herein, “TID”, “QD” and “QHS” have their ordinary meanings of “three times a day”, “once daily,” and “once before bedtime”, respectively.

Administration of an agent “in combination with” includes parallel administration (administration of both the agents to the patient over a period of time, such as administration of a monoclonal antibody and a peptide hormone such as an incretin hormone or analog on alternate days for one month), co-administration (in which the agents are administered at approximately the same time, e.g., within about a few minutes to a few hours of one another), and co-formulation (in which the agents are combined or compounded into a single dosage form suitable for oral, subcutaneous or parenteral administration).

“Hamster INGAP” is a non-human islet neogenesis associated peptide having the sequence Ile-GIy-Leu-His-Asp-Pro-Ser-His-Gly-Thr-Leu-Pro-Asn-Gly-Ser (SEQ ID NO: 1).

“HIP3” (Ile-Gly-Leu-His-Asp-Pro-Thr-Gln-Gly-Thr-Glu-Pro-Asn-Gly-Glu (SEQ ID NO: 2)) is a Human proIslet Peptide in purified, synthetic, or recombinant form. HIP3 has a molecular weight of about 1564.6.

“HIP2” (Ile-Gly-Leu-His-Asp-Pro-Thr-Gln-Gly-Thr-Glu-Pro-Asn-Gly (SEQ ID NO: 3)) is a Human proIslet Peptide in purified, synthetic, or recombinant form.

“HIP2” (Ile-Gly-Leu-His-Asp-Pro-Thr-Gln-Gly-Thr-Glu-Pro-Asn-Gly (SEQ ID NO: 4)) is a Human proIslet Peptide in purified, synthetic, or recombinant form, HIP2 has a molecular weight of about 1435.5.

HIP3Blocked or HIP3B (Ac-Ile-Gly-Leu-His-Asp-Pro-Thr-Gln-Gly-Thr-Glu-Pro-Asn-Gly-Glu-NH2) (SEQ ID NO: 5)) is a Human proIslet Peptide which has been blocked with a C-terminal acetyl group and an n-terminal amide group, in purified, synthetic, or recombinant form. HIPB has a molecular weight of about 1605.7.

HIP1Blocked (Ac-Trp-Ile-Gly-Leu-His-Asp-Pro-Thr-Gln-Gly-Thr-Glu-Pro-Asn-Gly-NH2 (SEQ ID NO: 6)) is a Human proIslet Peptide which has been blocked with a c-terminal acetyl group and an n-terminal amide group, in purified, synthetic, or recombinant form.

HIP2Blocked or HIP2B (Ac-Ile-Gly-Leu-His-Asp-Pro-Thr-Gln-Gly-Thr-Glu-Pro-Asn-Gly-NH2) (SEQ ID NO: 7)) is a Human proIslet Peptide which has been blocked with a c-terminal acetyl group and an n-terminal amide group, in purified, synthetic, or recombinant form. HIP2B has a molecular weight of about 1476.6.

HIP3Cys (Ile-Gly-Leu-His-Asp-Pro-Thr-Gln-Gly-Thr-Glu-Pro-Asn-Gly-Glu-Cys) (SEQ ID NO: 8)) is a Human proIslet Peptide which has an additional n-terminal cysteine residue, in purified, synthetic or recombinant form.

HIP1Cys (Trp-Ile-Gly-Leu-His-Asp-Pro-Thr-Gln-Gly-Thr-Glu-Pro-Asn-Gly-Cys) (SEQ ID NO: 9)) is a Human proIslet Peptide which has an additional n-terminal cysteine residue, in purified, synthetic or recombinant form.

HIP2Cys (Ile-Gly-Leu-His-Asp-Pro-Thr-Gln-Gly-Thr-Glu-Pro-Asn-Gly-Cys) (SEQ ID NO: 10)) is a Human proIslet Peptide which has an additional n-terminal cysteine, residue, in purified, synthetic or recombinant form.

HIP3CysDimer (Ile-Gly-Leu-His-Asp-Pro-Thr-Gln-Gly-Thr-Glu-Pro-Asn-Gly-Cys-Cys-Gly-Asn-Pro-Glu-Thr-Gly-Gln-Thr-Pro-Asp-His-Leu-Gly-Ile) (SEQ ID NO: 11) is a Human proIslet Peptide dimer wherein each monomer has been modified to include an n-terminal cysteine residue, in purified, synthetic, or recombinant form. The dimer forms via the creation of a disulfide bond between the cysteine residues of the individual monomers, as shown below:

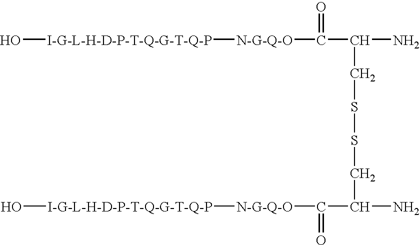

HIP1CysDimer (Trp-Ile-Gly-Leu-His-Asp-Pro-Thr-Gln-Gly-Thr-Glu-Pro-Asn-Gly-Glu-Cys-Cys-Glu-Gly-Asn-Pro-Glu-Thr-Gly-Gln-Thr-Pro-Asp-His-Leu-Gly (SEQ ID NO: 12)) is a Human proIslet Peptide dimer wherein each monomer has been modified to include an n-terminal cysteine residue, in purified, synthetic, or recombinant form. The dimer forms via the creation of a disulfide bond between the cysteine residues of the individual monomers, as shown below:

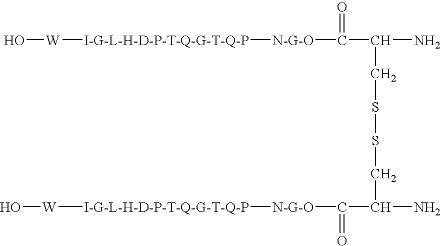

HIP2CysDimer (Ile-Gly-Leu-His-Asp-Pro-Thr-Gln-Gly-Thr-Glu-Pro-Asn-Gly-Cys-Cys-Gly-Asn-Pro-Glu-Thr-Gly-Gln-Thr-Pro-Asp-His-Leu-Gly-Ile) (SEQ ID NO: 13)) is a Human proIslet Peptide dimer wherein each monomer has been modified, to include an n-terminal cysteine residue, in purified, synthetic, or recombinant form. The dimer forms via the creation of a disulfide bond between the cysteine residues of the individual monomers, as shown below:

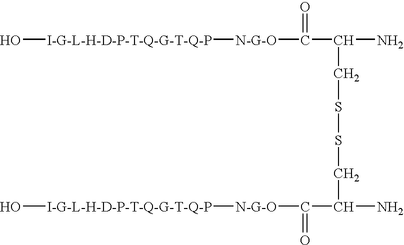

HIP3CysBlocked (Ac-Ile-GIy-Leu-His-Asp-Pro-Thr-Gln-Gly-Thr-Gln-Pro-Asn-Gly-Glu-Cys-NH2) (SEQ ID NO: 14)) is a Human proIslet Peptide which has been modified to include an n-terminal cysteine residue and has been blocked with a c-terminal acetyl group and an n-terminal amide group, in purified, synthetic, or recombinant, form.

HIP1CysBlocked (Ac-Trp-Ile-Gly-Leu-His-Asp-Pro-Thr-Gln-Gly-Thr-Glu-Pro-Asn-Gly-Cys-NH2) (SEQ ID NO: 15) is a Human proIslet Peptide which has been modified to include an n-terminal cysteine residue and has been blocked with a c-terminal acetyl group and an n-terminal amide group, in purified, synthetic, or recombinant form.

HIP2CysBlocked (Ac-Ile-Gly-Leu-His-Asp-Pro-Thr-Glu-Pro-Asn-Gly-Cys-NH2) (SEQ ID NO: 16) is a Human proIslet Peptide which has been modified to include an n-terminal cysteine residue and has been blocked with a c-terminal acetyl group and an n-terminal amide group, in purified, synthetic, or recombinant form.

HIP3CysBlockedDimer (2(Ac-Ile-Gly-Leu-His-Asp-Pro-Thr-Gln-Gly-Thr-Glu-Pro-Asn-Gly-Glu-Cys-NH2) (SEQ ID NO: 17) is a Human proIslet Peptide dimer wherein each monomer has been modified to include an n-terminal cysteine residue and has been blocked with a c-terminal acetyl group and an n-terminal amide group, in purified, synthetic, or recombinant form. The dimer forms via the creation of a disulfide bond between the cysteine residues of the individual monomers, as shown below:

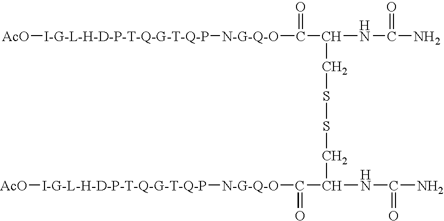

HIP1CysBlockedDimer (2(Ac-Trp-Ile-Gly-Leu-His-Asp-Pro-Thr-Gln-Gly-Thr-Glu-ProAsn-Gly-Cys-NH2)) (SEQ ID NO: 18) is a Human proIslet Peptide dimer wherein each monomer has been modified to include an n-terminal cysteine residue and has been blocked with a c-terminal acetyl group and an n-terminal amide group, in purified, synthetic, or recombinant form. The dimer forms via the creation of a disulfide bond between the cysteine residues of the individual monomers, as shown below:

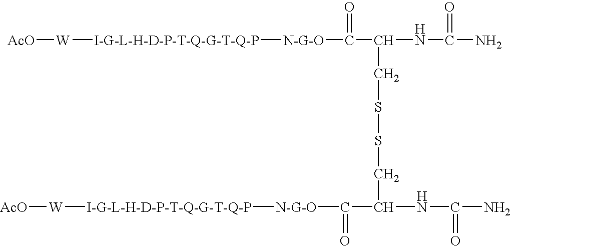

HIP2CysBlockedDimer or HIP2B Cys Dimer (2(Ac-Ile-Gly-Leu-His-Asp-Pro-Thr-Gln-Gly-Thr-Glu-Pro-Asn-Gly-Cys-NH2) (SEQ ID NO: 19) is a Human proIslet Peptide dimer wherein each monomer has been modified to include an n-terminal cysteine residue and has been blocked with a c-terminal acetyl group and an n-terminal amide group, in purified, synthetic, or recombinant form. The dimer forms via the creation of a disulfide bond between the cysteine residues of the individual monomers, as shown below:

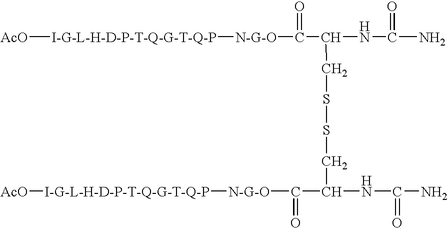

HIP2B Cys Dimer has a molecular weight of about 3157.5.

HIP3CysPEG (Ile-Gly-Leu-His-Asp-Pro-Thr-Gln-Gly-Thr-Glu-Pro-Asn-Gly-Glu-Cys-PEG) (SEQ ID NO: 20)) is a Human proIslet Peptide which has been modified to include an n-terminal cysteine residue to which has been covalently bonded to a dimeric maleimide activated 40 Kd PEG construct, in purified, synthetic, or recombinant form.

HIP1CysPEG (Trp-Ile-Gly-Leu-His-Asp-Pro-Thr-Gln-Gly-Thr-Glu-Pro-Asn-Gly-Cys-PEG) (SEQ ID NO: 21)) is a Human proIslet Peptide which has been modified to include an n-terminal cysteine residue to which has been covalently bonded to a dimeric maleimide activated 40 Kd PEG construct, in purified, synthetic, or recombinant form.

HIP2CysPEG (Ile-Gly-Leu-His-Asp-Pro-Thr-Gln-Gly-Thr-Glu-Pro-Asn-Gly-Cys-PEG) (SEQ ID NO: 22) is a Human proIslet Peptide which has been modified to include an n-terminal cysteine residue to which has been covalently bonded to a dimeric maleimide activated 40 Kd PEG construct, in purified, synthetic, or recombinant form.

HIP3CysBlockedPEG (Ac-Ile-Gly-Leu-His-Asp-Pro-Thr-Gln-Gly-Thr-Glu-Pro-Asn-Gly-Glu-Cys-PEG-NH2) (SEQ ID NO: 23) is a Human proIslet Peptide which has been blocked with a c-terminal acetyl group and an n-terminal amide group, and modified to include an n-terminal cysteine residue to which has been covalently bonded to a dimeric maleimide activated 40 Kd PEG construct, in purified, synthetic, or recombinant form as shown below:

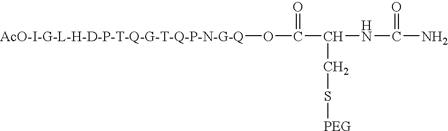

HIP1CysBlockedPEG (Ac-Trp-Ile-Gly-Leu-His-Asp-Pro-Thr-Gln-Gly-Thr-Glu-Pro-Asn-Giy-Cys-PEQ-NH2) (SEQ ID NO: 24) is a Human proIslet Peptide which has been blocked, with a c-terminal acetyl group and an n-terminal amide group, and modified to include an n-terminal cysteine residue to which has been, covalently bonded to a dimeric maleimide activated 40 Kd PEG construct, in purified, synthetic, or recombinant form.

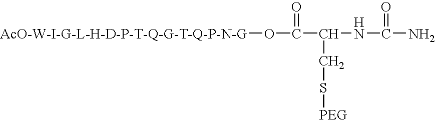

HIP2CysBlockedPEG or HIP2B Cys-PEG (Ac-Ile-Gly-Leu-His-Asp-Pro-Thr-Gln-Gly-Thr-Glu-Pro-Asn-Gly-Cys-PEG-NH2) (SEQ ID NO: 25) is a Human proIslet Peptide which has been blocked with a c-terminal acetyl group and an n-terminal amide group, and modified to include an n-terminal cysteine residue to which has been covalently bonded to a dimeric maleimide activated 40 Kd PEG construct, in purified, synthetic, or recombinant form. HIP2B Cys-PEG has a molecular weight of about 44,782.

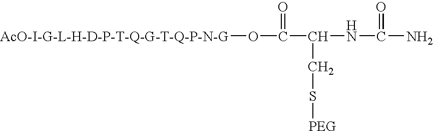

Since 1922, insulin has been the only available therapy for the treatment of type 1 diabetes and other conditions related to lack of or diminished production of insulin. It is well established mat at the onset of type 1 diabetes, patients have already lost at least 90% of their islets and their number of islets continues to steadily decline. However what has recently become clear is that not only in type 1 diabetes is there a deficit of islet mass, but also at the lime of diagnosis of type 2 diabetes, patients exhibit a loss of at least 50% of the islet mass and number. As with type 1 patients, the number and mass of islets continues to decline in type 2 diabetes, not from autoimmune attack, but because the beta cells effectively become “burned out.” Although this decline occurs more rapidly in type 1 patients, there is still a decline of 10-20% per year among type 2 patients.

A common misunderstanding is that insulin resistance causes type 2 diabetes. Although insulin resistance is a feature of both diabetes and obesity, diabetes does not occur as a result of insulin resistance without the coexistence of reduction of islet mass leading to reduction in insulin secretion. Diabetes occurs only when there is a critical reduction in islet mass and function that prevents an adequate insulin response to a carbohydrate stimulus. Despite some regenerative ability of the beta cells within the islet structures, the islets have a much slower ability to regenerate.

Even in the face of blocking the autoimmune destruction of the insulin-producing cells in the pancreas, without new methods of regenerating islets, there will not be an end to type 1 diabetes.

Loss of islet mass is the basis of both type 1 and 2 diabetes, and more recent studies have demonstrated that prediabetes, insulin resistant states, hypertension, inactivity and family history are islet stressors with diminished islet mass.

Despite decades of research and the advent of pancreatic islet cell transplantation and newer claims of success resulting from the Edmonton Protocol for islet cell transplantation, the success has not been replicated in the United States. At four years post-transplant, fewer than 10% of patients who have received islet cell transplants remain insulin independent. Additionally, despite new immune suppression protocols, there is an 18% rate per patient of serious side effects.

In a normally functioning pancreas, small numbers of islets die naturally on a day-by-day basis and are replaced as required to keep glucose levels under control. On average, this regenerative process known as islet neogenesis replaces islets at a rate of approximately 2% per month. In nondiabetic patients, the beta cell mass within the existing islets can expand or contract depending on the insulin needs of the individual. This process is referred to as “beta cell proliferation” does not occur in patients with type 1 diabetes and is limited in type 2 patients.

The study of islet neogenesis is not new. In 1920, it was reported that an obstructive pancreatic stone resulted in atrophy to most of the pancreas but an increase in islets. It was then hypothesized that ligating (binding) the pancreatic duets might lead to the identification of a substance that could be useful in the treatment of diabetes. Nearly a centure ago, based upon autopsy findings from fatal pancreatic stones with the result being islet proliferation, surgeons in the early 1900s ligated the pancreatic tail of diabetic children in the hopes of producing substances that would form new islets. Although the positive effects of these procedures were short-lived, they demonstrated the potential for islet restoration in humans.

Pancreatic ligation studies that were intended to create a hamster model for pancreatitis resulted in the formation of many new islets. This research led to the isolation of a hamster peptide referred to as the Islet Neogenesis Associated Peptide, or INGAP. In the clinical development of INGAP, it was further demonstrated that new human islets could be differentiated from the stem-cell-like islet progenitor cells that reside throughout the adult pancreas even decades after the onset of type 1 diabetes.

Separate from the concept of using pancreatic ligation to produce new islets, regeneration of islets during pregnancy has been described. Islets are formed in late embryogenesis and pregnancy data demonstrates the islet population grows postnatally. Research has demonstrated that islet neogenesis precedes beta cell expansion during pregnancy. Furthermore, it has been described that postnatally, in humans, there are precursor cells within the pancreas, that are capable of expansion occur naturally and efficiently differentiate into clusters of islets.

The primary way in which patients with type 1 or later-stage type 2 diabetes manage their disease is by administering insulin, either via subcutaneous injection or by using a subcutaneous pump infusion. As well as the obvious lifestyle disadvantages, insulin therapy does not match the body's normal glucose control mechanisms and therefore does not fully manage glucose fluctuations. Even the best-controlled type 1 diabetic patients do not have anything remotely like a normal glucose metabolism. This is because insulin secretion is only part of the missing pancreatic function.

Over the past several decades, there have been several new therapies have become available for diabetes, which may improve metabolic function of the existing beta cells or islets within the pancreas. These are agents which may improve existing islet function, and glucose metabolism include: Glucagon Like Peptide-1 (GLP-1) and its analogs. Gastric Inhibitory Peptide/Glucose-Dependent Insulinoptropic polypeptide (GIP), Amylin, and its analog, Pramlintide, and GLP-1 receptor agonists, such as Liraglutide (NN2211) and Exendin-4, or compounds which halt the destruction of GLP-1, such as Dipeptidyl Peptidase-4 Inhibitors, (DPP-4 inhibitors), and including but not limited to Vildagliptin, Sitagliptin, Saxagliptin, and PHX1149. Other compounds which may improve existing islet function include: gastrin, epidermal growth factor-1 and insulin sensitizing agents including the biguanide. Metformin, and the thiazolidinediones, Rosiglitazone and Pioglitazone. Other agents that may impact pancreatic function that may be utilized with the islet cell neogenesis agent include AGI-1067, an anti-inflammatory antioxidant agent that, works by inhibiting signaling pathways that are activated in response to oxidative stress and pro-inflammatory stimuli, Rimonabant and other drugs that block the cannabinoid receptor 1 (CB1), gut peptide, PYY, inclusive of, but not limited to PYY3-36 (PYY) nasal spray, the hypothalamic neuropeptide Y (NPY) and drugs that impact the leptin, ghrelin, pro-opiomelanocortin/melanocortin pathways or the melanocortin receptor, orlistat, sibutramine or acarbose.

Proof of the elasticity of the pancreas with respect to the generation of new pancreatic cells throughout one's lifetime accompanied by pancreatic cell death or apoptosis has replaced the long held concept that, the number of insulin producing islet cells is fixed at birth and sustained throughout life. It is currently accepted that pancreatic islet cell neogenesis occurs from progenitor cells that exist within the adult pancreas. Studies confirm that progenitor cells exist within both the islet and ductal fractions of the adult human pancreas, and that upon stimulation with HIP, there is both increased insulin production along with islet numbers. This supports the data on pancreatic plasticity during pregnancy where studies among type 1 women, as many as ⅓ of women have a dramatic, reduction in insulin requirements, with some women coming off insulin completely during their pregnancy. Even among patients who have had type 1 diabetes for decades, during pregnancy, many secrete normal levels of (C-peptide, when C-peptide was non-detectable on the onset of pregnancy. Similarly, patients with type 1 diabetes having received renal transplants and on long term immunosuppression have been observed to regenerate insulin producing islet.

Additionally, over the past decade, clinical trials have been conducted to evaluate the impact of a number of immune modulators that may arrest the destruction of the pancreas. The studies and types of agents to potentially arrest the destruction of islet cells have varied considerably. The types of agents include general immunosuppressant agents which have typically been used in organ transplants, specifically targeted antibodies to those lymphocytes which attack the islets, along with other agents such as Vitamin D, in which a deficiency has been associated with a higher incidence of diabetes.

Anti CD-3 antibodies that target the immune response and specifically block the T-lymphocytes that cause islet cell death in type 1 diabetes have been utilized as well as heat-shock proteins to arrest the destruction of insulin-producing cells and anti-GAD65 antibody vaccines. Trials are underway with a number of diverse agents or combination of agents among newly diagnosed patients with diabetes. Currently the immune agents mycophenolate mofetil, Rituximab, an anti CD20 agent, which is an FDA approved agent for the treatment of B-lymphocyte lymphoma, is also being studied in the preservation of islet cells among newly diagnosed type 1 diabetes patients.

Trials are underway in newly diagnosed type 1 diabetes patients using the anti CD3 antibody, hOKT3 gamma1 (Ala-Ala), also known as MGA031 and the monoclonal antibody TRX4 (ChAglyCD3). The immune tolerance agent may also include, Polyclonal Anti-T-Lymphocyte Globulin (ATG), CTLA4-Ig (Abatacept) a selective costimulation modulator as it inhibits the costimulation of T cells, Campath-1H, (Anti-CD52 Antibody), a humanized monoclonal antibody to T-cells, Polyclonal Anti-T-Lymphocyte Globulin (ATG), DiaPep277, a derivative Heat Shock Protein 60, that may activate a subgroup of T-cells, which down-regulate T lymphocytes, anti-GAD antibody vaccine based on the 65 kDa isoform of the recombinant human glutamic acid decarboxylase protein (rhGAD65). DiaPep277 is another immune tolerance agent directed at the onset of type 1 diabetes to halt the destruction of islets. DiaPep277 is a heat shock protein, which is believed to impact the release of cytokines and pro-inflammatory cells which destroy islet cells, is being studied in adults and children with newly diagnosed patients with diabetes and also in patients with Latent Autoimmune Diabetes in Adults (LADA). CTLA4-Ig (Abatacept) inhibits a crucial stimulatory pathway in the activation of T cells. By this mechanism, the drug is thought to arrest or slow the T cell mediated autoimmune destruction of insulin producing cells and preserve their function. CTLA-4-Ig is being trialed as an intravenous agent begun within three months of diagnosis and then monthly for a total of 25 treatments. CampathH1 is another immune tolerance, agent being trialed among-new onset type 1 diabetes and may be utilized in conjunction with HIP, Optimized HIP, hamster INGAP and other islet neogenesis agents for improvement in type 1 diabetes.

The aim of all of the therapies that are proposed to prevent further immune destruction of the islet cells while, enhancing further transformation of new islets, which is a very slow process. Thus, immune therapy alone, even when delivered to newly diagnosed type 1 diabetes patients, has not been able to render patients insulin-free. Typically, a healthy individual requires about 1.5 million islet cells to maintain glucose homeostatsis. At the time of diagnosis, both type 1 and type 2 patients only retain about 50% or less of their typical islet cell mass (type 1 patients retain 10% or less of their insulin-producing cell function, while type 2 patients retain about 50% of their insulin-producing cell function). This ongoing destructive process in type 1 diabetes is typically more rapid and progressive than in type 2 diabetes leading to multiple daily insulin injections to survive. The typical, healthy adult has an estimated cell death rate for islets of between 1000 and 2000 cells per day; the human islet lifespan is estimated at about 3 years. Each day, the same number of new islets are formed from precursor cells within the pancreas, both in the endocrine and exocrine portions of the organ. Thus, even if immune-halting agents are used to prevent, further islet loss, because the dally regeneration rate of new islet production is only about 0.1% per day, it could take years, if not decades, to repopulate the pancreas with insulin producing cells without such an immune-blocking compound being combined with a regeneration compound such as Human, proIslet Peptide (HIP), Optimized HIP, hamster INGAP or other islet neogenesis agents.

One embodiment of the present invention provides a method for treating newly diagnosed or pre-existing type 1 diabetes mellitus in a patient, said method comprising administering to said patient of an agent that stimulates pancreatic islet cell regeneration and/or transformation of new insulin producing islets in combination with an immune tolerance agent or combination of immune tolerance agents. An agent that stimulates pancreatic islet cell regeneration and/or transformation of new insulin producing islets includes, but is not limited to, Human proIslet Peptide (HIP), Optimized HIP, hamster INGAP other islet neogenesis agents. Preferably, the islet neogenesis agent is selected from HIP and Optimized HIP, preferably HIP2 and Optimized HIP2, such as HIP2B. Immune tolerance agents include, but are not limited to, mycophenolate mofetil, daclizumab, rituximab (anti CD20), anti CD3 antibodies including hOKT3 gamma 1 (Ala-Ala), also known as MGA031 and the monoclonal antibody TRX4 (ChAglyCD3), CTLA4-Ig (abatacept) a selective costimulation modulator as it inhibits the costimulation of T cells, campath-1H, anti-CD52 antibody, a or humanized monoclonal antibody to T-cells, polyclonal anti-T-lymphocyte globulin (ATG), DiaPep277, anti-GAD antibody vaccine based on the 65 kDa isoform of the recombinant human glutamic acid decarboxylase protein (rhGAD65), and diazoxide. Preferably, each agent is administered at a therapeutically effective amount.

The combination of therapies may restore more normal glucose metabolism, including achieving and maintaining appropriate levels of insulin, amylin, glucagon, somatostatin and pancreatic polypeptides that are normally secreted from islets among patients without diabetes. By restoring normal islet function, and protecting the newly form islets, there will, in turn, be improvement in premeal and postprandial glucose levels, hemoglobin AlC, triglycerides, and glucagon and ameliorate the significant weight gain and increased risk for serious hypoglycemia that has been associated with tight glycemic control utilizing exogenous insulin among insulin-requiring patients, whether they have type 1 or type 2 diabetes.

In certain, embodiments, the method may further comprise administering Vitamin D or a derivative thereof, including, but not limited, to cholecalciferol and 1,25 dihydroxyvitamin D.

In certain embodiments, the method may further comprise administering a beta cell or islet function optimizing agent. Such agents include, but are not limited to, Glucagon Like Peptide-1 (GLP-1) and its analogs. Gastric Inhibitory Peptide/Glucose-Dependent Insulinoptropic polypeptide (GIP), Amylin, and its analog, Pramlintide, and GLP-1 receptor agonists, such as Liraglutide (NN2211) and Exendin-4/exenatide, or compounds which halt the destruction of GLP-1, such as Dipeptidyl Peptidase-4 inhibitors, (DPP-4 inhibitors), including but not limited to Vildagliptin, Sitagliptin, Saxagliptin, and PHX1149. Other compounds which may improve existing islet function include: gastrin, epidermal growth factor-1 and insulin sensitizing agents including the thiazolidinediones, including but not limited to Rosiglitazone and Pioglitazone, AGI-1067, an anti-inflammatory antioxidant agent that works by inhibiting signaling pathways that are activated in response to oxidative stress and pro-inflammatory stimuli, Rimonabant and other drugs that block, the cannabinoid receptor 1 (CB1), gut peptide, PYY, inclusive of, but not limited to PYY3-36 (PYY) nasal spray, the hypothalamic neuropeptide Y (NPY) and drugs that impact the leptin, ghrelin, pro-opiomelanocortin/melanocortin pathways or the melanocortin receptor, orlistat, which impacts the gut, centrally acting sibutramine, or acarbose, which delays carbohydrate absorption along the brush border of the intestine. The additional therapy may be beneficial, particularly in patients with type 1 diabetes who are above their ideal body weight and predisposed to peripheral insulin resistance.

The methods are particularly efficacious, because, unlike prior therapies, the therapeutic methods of the invention uniquely promote islet cell regeneration while also inhibiting the autoimmune cells that caused the destruction of the islet, cells and therefore the insulin dependency of the patient.

In a further embodiment, the first dosage of the immune tolerance agent or agents may be administered 8-12 weeks prior to initiation of HIP. Optimized HIP, hamster INGAP, or other islet neogenesis agents. Four to eight weeks following the administration of the immune tolerance agent or agents, depending on the immune and overall health status of the patient, there may be a 4-week period in which patients will intensify their glycemic status.

During this period of optimization of glycemic control, the glucose goal for patients may be between 100 and 200 mg/dL at all times. In order to achieve the optimized glucose goals to initiate HIP, Optimized HIP or other islet neogenesis agents, patients may utilize a medical team with state-of-the-art diabetes tools including subcutaneous continuous monitoring systems. The primary goal will be to ensure that the glucose levels do not fall below 70 mg/dL during the optimization period.

There may be a two week period prior to the initial administration of HIP, Optimized HIP, hamster INGAP or other islet neogenesis agents, in which the patients should not have any episodes of symptomatic hypoglycemia. Should patients exhibit symptomatic hypoglycemia once HIP, Optimized HIP, hamster INGAP or other islet neogenesis agents is administered, the patients' diabetes regimen should be modified because hypoglycemia may negate the effects of all islet neogenesis compounds including HIP. Optimized HIP, hamster INGAP or other islet neogenesis agents.

HIP, Optimized HIP, hamster INGAP or other islet neogenesis agents may be delivered within 15 minutes of the major meals eaten throughout the day or night, preferably when at least 30 grams of carbohydrates are consumed. These agents may be delivered via subcutaneous injection, continuous subcutaneous pump delivery or orally. For example, the dosage of HIP and Optimized HIP range from 5-150 mg/kg/day, preferably with each major meal, depending on whether the preparation is oral or subcutaneous and depending upon the selected HIP or Optimized HIP peptide delivered.

During the period in which HIP, Optimized HIP, hamster INGAP or other islet neogenesis agents are administered, the patients' insulin dosage may be decreased as required to prevent any episodes of hypoglycemia and to maintain glucose levels in an optimal range for islet neogenesis and may initially be reduced by 1% per day for the first 30 days. This is a total reduction of 1% per day from the preprandial insulin dosages (0.33% per meal reduction of the total premeal insulin dosage from the previous premeal dosage). During about days 31-60 on HIP, Optimized HIP, hamster INGAP or other islet neogenesis agents, there may be a 1% per day reduction in the basal insulin from the previous day.

During the first about 60 days of islet neogenesis therapy, patients may have daily communication via phone, e-mail or office visits to give feedback on glucose values to the diabetes health care team. Based on the glucose values, more aggressive reduction in basal insulin dosages may occur if premeal glucose levels are less than 100 mg/dL and more aggressive reductions in premeal insulin may occur if 2 hour postprandial levels are less than 140 mg/dL.

During about days 61-90 on islet neogenesis agent therapy, insulin dosages maybe reduced by 0.5-2.0% per day based upon daily glucose values. Reduction in basal insulin dosages may be required to prevent any episodes of hypoglycemia and to maintain glucose levels in an optimal range for islet neogenesis and may initially be reduced by 0.5% per day if premeal values are 100-125 mg/dL and a 0.6% per day total reduction (0.18% per meal reduction from the previous day) in premeal insulin may occur if 2 hour postprandial levels are 140-160 mg/dl. Each day there may be a 1% reduction in basal insulin if premeal glucose levels are less than 100 mg/dL and % reduction (0.33% per meal reduction from the previous day) in premeal insulin may occur if 2 hour postprandial levels are less than 140 mg/dL. If there are episodes of hypoglycemia in the premeal period, there may be a reduction of 2.0% from the previous day in basal insulin from the previous day. If there are any episodes of hypoglycemia during the postprandial phase, the dosage of preprandial insulin may be reduced by 2.0% before meals (0.7% per meal in premeal insulin).

Islet neogenesis therapy with HIP, Optimized HIP, hamster INGAP or other islet neogenesis can be discontinued when stimulated C-peptide levels are within the normal range and when optimized glycemic control has been achieved without the usage of other diabetic agents including insulin.

Throughout the duration of the treatment patients may have daily communication via phone, e-mail or office visits to give feedback on glucose values to the diabetes health care team. Based on the glucose values, more aggressive reduction in basal and premeal insulin dosages may occur based on premeal and prostprandial glucose levels respectively.

The immune tolerance agents to be administered about 8-12 weeks prior to administration of islet neogenesis therapy with HIP, Optimized HIP, hamster INGAP or other islet neogenesis may include: Mycophenolate mofetil, Daclizumab, Rituximab (and CD20), anti CD3 antibodies including hOKT3 gamma 1 (Ala-Ala), also known as MGA031 and the monoclonal antibody TRX4 (ChAglyCD3), CTLA4-Ig (Abatacept) a selective costimulation modulator as it inhibits the costimulation of T cells. Campath-1H, Anti-CD52 Antibody, a or humanized monoclonal antibody to T-cells, Polyclonal Anti-T-Lymophocyte Globulin (ATG). DiaPep277, anti-GAD antibody vaccine based on the 65 kDa isoform of the recombinant human glutamic acid decarboxylase protein (rhGAD65), and other approaches to immune suppression including bedtime Diazoxide.

In addition. Vitamin D and Vitamin D derivatives including, but not limited to cholecalciferol, 1,25 dihydroxyvitamin D may further be administered. For example, 2000-4000 IU of Vitamin D3, cholecalciferol, may be given daily beginning 12 weeks prior to initiation of islet neogenesis therapy with HIP, Optimized HIP, hamster INGAP or other islet neogenesis agents. Vitamin D3 may be delivered orally daily throughout the glucose optimization phase and throughout delivery of islet neogenesis therapy with HIP, Optimized FOP, hamster INGAP or other neogenesis agents, based upon the serum levels of 25 hydroxy vitamin D levels to optimize levels of between 45-50 ng/ml or 115-128 nmol/l.

In another embodiment, in addition to optimizing Vitamin D3 levels to protect new islets, the immune tolerance agent Polyclonal Anti-T-Lymphocyte Globulin (ATG) may be initiated 8-12 weeks prior to initiation of HIP, Optimized HIP, hamster INGAP or other islet neogenesis agents. In such an embodiment, about four dosages of ATG doses may be given. The first dosage of ATG may be about 9 mg/kg of body weight, then 3 consecutive doses of about 3 mg/kg may be administered intravenously over 4 hours. One hour before the first ATG administration, a cutaneous tolerance test (0.2 ml of the final solution) may be performed. In one embodiment of the combination therapy, ATG may be delivered prior to the usage of Optimized HIP or Optimized HIP analog or derivative. A second administration of the ATG may be required based on quarterly measurements of anti-GAD65 antibodies and other immune markers suggesting autoimmune attack at 24 months after the initial treatment with ATG. Earlier treatment may be required if there is a significant rise in autoimmune antibodies directed toward the pancreas.

Following the initiation of ATG, based on the immune and health profile, the patient may have 4 weeks of optimizing their glycemic control and HIP, Optimized HIP, hamster INGAP or another islet neogenesis agent is initiated. During this period of optimization of glycemic control, the glucose goal for patients may be between 100 and 200 mg/dL. In order to achieve the optimized glucose goals to initiate HIP, Optimized HIP or other islet neogenesis agents, patients may utilize a medical team with state-of-the-art diabetes tools including subcutaneous continuous monitoring systems. The primary goal will be to ensure that no glucose levels falls below 70 mg/dL during the optimization period. There should be a two week period prior to the initial administration of HIP, Optimized HIP, hamster INGAP or other islet neogenesis agents, in which the patients should not have any episodes of symptomatic hypoglycemia. Should patients exhibit symptomatic hypoglycemia once HIP, Optimized HIP, hamster INGAP or other islet neogenesis agents is administered, the patients' diabetes regimen may be modified because hypoglycemia may negate the effects of all islet neogenesis compounds including HIP, Optimized HIP, hamster INGAP or other islet neogenesis agents.

HIP, Optimized HIP, hamster INGAP or other islet neogenesis agents may delivered within 15 minutes of the major meals eaten throughout the day or night, preferably when at least 30 grams of carbohydrates are consumed. These agents may be delivered via subcutaneous injection, continuous subcutaneous pump delivery or orally. For example, the dosage of HIP and Optimized HIP range from 5-150 mg/kg/day, preferably with each major meal, depending on whether the preparation is oral or subcutaneous and depending upon the selected HIP or Optimized HIP peptide delivered.

During the period in which HIP, Optimized HIP hamster INGAP or other islet neogenesis agents are administered, the patients' insulin dosage may be decreased as required to prevent any episodes of hypoglycemia and to maintain glucose levels in an optimal range for islet neogenesis and may initially be reduced by 1% per day for the first about 30 days. This is a total reduction of 1% per day from the preprandial insulin dosages (0.33% per meal reduction of the total premeal insulin dosage from the previous premeal dosage). During about days 31-60 on HIP, Optimized HIP, hamster INGAP or other islet neogenesis agents, there may be a 1% per day reduction in the basal insulin from the previous day.

During the first 60 days of islet neogenesis therapy, patients will have daily communication via phone, e-mail or office visits to give feedback on glucose values to the diabetes health care team. Based on the glucose values, more aggressive reduction in basal insulin dosages will occur if premeal glucose levels are less than 100 mg/dL and more aggressive reductions in premeal insulin will occur if 2 hour postprandial levels are less than 140 mg/dL.

During about days 61-90 on islet neogenesis agent therapy, insulin dosages maybe reduced by 0.5-2.0% per day based upon daily glucose values. Reduction in basal insulin dosages may be required to prevent any episodes of hypoglycemia and to maintain glucose levels in an optimal range for islet neogenesis and may initially be reduced by 0.5% per day if premeal values are 100-125 mg/dL and a 0.6% per day total reduction (0.18% per meal reduction from the previous day) in premeal insulin may occur if 2 hour postprandial levels are 140-160 mg/dl. Each day there may be a 1% reduction in basal insulin if premeal glucose levels are less than 100 mg/dL and 1% reduction (0.33% per meal reduction from the previous day) in premeal insulin may occur if 2 hour postprandial levels are less than 140 mg/dL. If there are episodes of hypoglycemia in the premeal period, there may be a reduction of 2.0% from the previous day in basal insulin from the previous day. If there are any episodes of hypoglycemia during the postprandial phase, the dosage of preprandial insulin may be reduced by 2.0% before meals (0.7% per meal in premeal insulin).

In another embodiment, in addition to optimizing 25 hydroxy vitamin D levels to protect new islets, the immune tolerance agent mycophenolate mofetil (MMF) may be initiated 8-12 weeks prior to initiation of HIP, Optimized HIP, hamster INGAP or other islet neogenesis agents. MMF is given by mouth twice a day for two years.

Following the initiation of MMF, based on the immune and health profile, the patient may have 4 weeks of optimizing their glycemic control and HIP, Optimized HIP, hamster INGAP or another islet neogenesis agent is initiated. During this period of optimization of glycemic control, the glucose goal for patients may be between 100 and 200 mg/dL. In order to achieve the optimized glucose goals to initiate HIP, Optimized HIP or other islet neogenesis agents, patients may utilize a medical team with state-of-the-art diabetes tools including subcutaneous continuous monitoring systems. The primary goal will be to ensure that no glucose levels falls below 70 mg/dL during the optimization, period.

There may be a two week period prior to the initial administration of HIP, Optimized HIP, hamster INGAP or other islet neogenesis agents, in which the patients should not have any episodes of symptomatic hypoglycemia. Should patients exhibit symptomatic hypoglycemia once HIP, Optimized HIP, hamster INGAP or other islet neogenesis agents is administered, the patients' diabetes regimen should be modified because hypoglycemia will negate the effects of all islet neogenesis compounds including HIP, Optimized HIP, hamster INGAP or other islet neogenesis agents.

HIP, Optimized HIP, hamster INGAP or other islet neogenesis agents may be delivered within 15 minutes of the major meals eaten, throughout the day or night, preferably when the meal contains at least 30 grams of carbohydrates. These agents may be delivered via subcutaneous injection, continuous subcutaneous pump delivery or orally. For example, the dosage of HIP and Optimized HIP range from 5-150 mg/kg/day, preferably with each major mead depending on whether the preparation is oral or subcutaneous and depending upon the selected HIP or Optimized HIP peptide delivered.

During the period in which HIP, Optimized HIP, hamster INGAP or other islet neogenesis agents are administered, the patients' insulin dosage may be decreased as required to prevent any episodes of hypoglycemia and to maintain, glucose levels in an optimal range for islet neogenesis and may initially be reduced by 1% per day for the first about 30 days. This is a total reduction of 1% per day from the preprandial insulin dosages (0.33% per meal reduction of the total premeal insulin dosage from the previous premeal dosage). During days about 31-60 on HIP, Optimized HIP, hamster INGAP or other islet neogenesis agents, there may be a 1% per day reduction in the basal insulin from the previous day.

During the first about 60 days of islet neogenesis therapy, patients may have dally communication via phone, e-mail or office visits to give feedback on glucose values to the diabetes health care team. Based on the glucose values, more aggressive reduction in basal insulin dosages may occur if premeal glucose levels are less than 100 mg/dL and more aggressive reductions in premeal insulin may occur if 2 hour postprandial levels are less than 140 mg/dL.

During about days 61-90 on islet neogenesis agent therapy, insulin dosages maybe reduced by 0.5-2.0% per day based upon daily glucose values. Reduction in basal, insulin dosages may be required to prevent any episodes of hypoglycemia and to maintain glucose levels in an optimal range for islet neogenesis and may initially be reduced by 0.5% per day if premeal values are 100-125 mg/dL and a 0.6% per day total reduction (0.18% per meal reduction from the previous day) in premeal insulin may occur if 2 hour postprandial levels are 140-160 mg/dl. Each day there may be a 1% reduction in basal insulin if premeal glucose levels are less than 100 mg/dL and 1% reduction (0.33% per meal reduction from the previous day) in premeal insulin may occur if 2 hour postprandial levels are less than 140 mg/dL. If there are episodes of hypoglycemia in the premeal period, there may be a reduction of 2.0% from the previous day in basal insulin from the previous day. If there are any episodes of hypoglycemia during the postprandial phase, the dosage of preprandial insulin may be reduced by 2.0% before meals (0.7% per meal in premeal insulin).

In another embodiment, in addition to optimizing 25 hydroxy vitamin D levels to protect new islets, the immune tolerance agent daclizumab (DZB) will be initiated 8-12 weeks prior to initiation of HIP, Optimized HIP hamster INGAP or other islet neogenesis agents. DZB is given by an intravenous infusion twice, once at the time of enrollment and again about two weeks later.

Following the initiation of DZB, based on the immune and health profile, the patient may have 4 weeks of optimizing their glycemic control and HIP, Optimized HIP, hamster INGAP or another islet neogenesis agent is initiated. During this period of optimization of glycemic control, the glucose goal for patients may be between 100 and 200 mg/dL. In order to achieve the optimized glucose goals to initiate HIP, Optimized HIP or other islet neogenesis agents, patients may utilize a medical team with state-of-the-art diabetes tools including subcutaneous continuous monitoring; systems. The primary goal will be to ensure that no glucose levels falls below 70 mg/dL during the optimization period. There may be a two week-period prior to the initial administration of HIP, Optimized HIP, hamster INGAP or other islet neogenesis agents, in which the patients should not have any episodes of symptomatic hypoglycemia. Should patients exhibit symptomatic hypoglycemia once HIP, Optimized HIP, hamster INGAP or other islet neogenesis agents is administered, the patients' diabetes regimen may be modified because hypoglycemia may negate the effects of all islet neogenesis compounds including HIP, Optimized HIP, hamster INGAP or other islet neogenesis agents.

HIP, Optimized HIP, hamster INGAP or other islet neogenesis agents may delivered within 15 minutes of the major meals eaten throughout the day or night, preferably when the meal contains at least 30 grams of carbohydrates. These agents may be delivered via subcutaneous injection, continuous subcutaneous pump delivery or orally. For example, the dosage of HIP and Optimized HIP range from 5-150 mg/kg/clay, preferably with each major meal, depending on whether the preparation is oral or subcutaneous and depending upon the selected HIP or Optimized HIP peptide delivered.

During the period in which HIP, Optimized HIP, hamster INGAP or other islet neogenesis agents are administered, the patients' insulin dosage may be decreased as required to prevent any episodes of hypoglycemia and to maintain glucose levels in an optimal range for islet neogenesis and that initially be reduced by 1% per day for the first about 30 days. This is a total reduction of 1% per day from the preprandial insulin dosages (0.33% per meal reduction of the total premeal insulin dosage from the previous premeal dosage). During days about 31-60 on HIP, Optimized HIP, hamster INGAP or other islet neogenesis agents, there may be a 1% per day reduction in the basal insulin from the previous day.

During the first about 60 days of islet neogenesis therapy, patients may have daily communication via phone, e-mail or office visits to give feedback on glucose values to the diabetes health care team. Based on the glucose values, more aggressive reduction in basal insulin dosages may occur if premeal glucose levels are less than 100 mg/dL and more aggressive reductions in premeal insulin, may occur if 2 hour postprandial levels are less than 140 mg/dL.

During about days 61-90 on islet neogenesis agent therapy, insulin dosages maybe reduced by 0.5-2.0% per day based upon daily glucose values. Reduction in basal insulin dosages may be required to prevent any episodes of hypoglycemia and to maintain glucose levels in an optimal range for islet neogenesis and may initially be reduced by 0.5% per day if premeal values are 100-125 mg/dL and a 0.6% per day total reduction (0.18% per meal reduction from the previous day) in premeal insulin may occur if 2 hour postprandial levels are 140-160 mg/dl. Each day there may be a 1% reduction in basal insulin if premeal glucose levels are less than 1.00 mg/dL and 1% reduction (0.33% per meal reduction from the previous day) in premeal insulin may occur if 2 hour postprandial levels are less than 140 mg/dL. If there are episodes of hypoglycemia in the premeal period, there may be a reduction of 2.0% from the previous day in basal insulin from the previous day. If there are any episodes of hypoglycemia during the postprandial phase, the dosage of preprandial insulin may be reduced by 2.0% before meals (0.7% per meal in premeal insulin).

In another embodiment, in addition to optimizing 25 hydroxy vitamin D levels to protect new islets, the immune tolerance agents mycophenolate mofetil (MMF) and daclizumab (DZB) will be initiated 8-12 weeks prior to initiation of HIP, Optimized HIP, hamster INGAP or other islet neogenesis agents. DZB is given by an intravenous infusion twice, once at the time of enrollment and again about two weeks later. MMF is given by mouth twice a day for two years. Both agents may be initiated at the same time.

Following the initiation of MMF and DZB, based on the immune and health profile, the patient may have 4 weeks of optimizing their glycemic control and HIP, Optimized HIP, hamster INGAP or another islet neogenesis agent is initiated. During this period of optimization of glycemic control, the glucose goal for patients may be between 100 and 200 mg/dL. In order to achieve the optimized glucose goals to initiate HIP, Optimized HIP or other islet neogenesis agents, patients may utilize a medical team with state-of-the-art diabetes tools including subcutaneous continuous monitoring systems. The primary goal will be to ensure that no glucose levels falls below 70 mg/dL during the optimization period. There may be a two week period prior to the initial administration of HIP, Optimized HIP, hamster INGAP or other islet neogenesis agents, in which the patients should not have, any episodes of symptomatic hypoglycemia. Should patients exhibit symptomatic hypoglycemia once HIP, Optimized HIP, hamster INGAP or other islet neogenesis agents is administered, the patients' diabetes regimen may be modified because hypoglycemia may negate the effects of all islet neogenesis compounds including HIP, Optimized HIP, hamster INGAP or other islet neogenesis agents.

HIP, Optimized HIP, hamster INGAP or other islet neogenesis agents may be delivered within 15 minutes of the major meals eaten throughout the day or night, preferably when at least 30 grams of carbohydrates are consumed per meal. These agents may be delivered via subcutaneous injection, continuous subcutaneous pump delivery or orally. For example, the dosage of HIP and Optimized HIP range from 5-150 mg/kg/day, preferably with each major meal, depending on whether the preparation is oral or subcutaneous and depending upon the selected HIP or Optimized HIP peptide delivered.

During the period in which HIP, Optimized HIP, hamster INGAP or other islet neogenesis agents are administered, the patients' insulin dosage may be decreased as required to prevent any episodes of hypoglycemia and to maintain glucose levels in an optimal range for islet neogenesis and may initially be reduced by 1% per day for the first about 30 days. This is a total reduction of 1% per day from the preprandial insulin dosages (0.33% per meal reduction of the total premeal insulin dosage from the previous premeal dosage). During about days 31-60 on HIP, Optimized HIP, hamster INGAP or other islet neogenesis agents, there may be a 1% per day reduction in the basal insulin from the previous day.

During the first about 60 days of islet neogenesis therapy, patients may have daily communication via phone, e-mail or office visits to give feedback on glucose values to the diabetes health care team. Based on the glucose values, more aggressive reduction in basal insulin dosages may occur if premeal glucose levels are less than 100 mg/dL and more aggressive reductions in premeal insulin may occur if 2 hour postprandial levels are less than 140 mg/dL.

During about days 61-90 on islet neogenesis agent therapy, insulin dosages maybe reduced by 0.5-2.0% per day based upon daily glucose values. Reduction in basal insulin dosages may be required to prevent any episodes of hypoglycemia and to maintain glucose levels in an optimal range for islet neogenesis and may initially be reduced by 0.5% per day if premeal values are 100-125 mg/dL and a 0.6% per day total reduction (0.18% per meal reduction from the previous day) in premeal insulin, may occur if 2 hour postprandial levels are 140-160 mg/dl. Each day there may be a 1% reduction in basal insulin if premeal glucose levels are less than 100 mg/dL and 1% reduction (0.33% per meal reduction from the previous day) in premeal insulin may occur if 2 hour postprandial levels are less than 140 mg/dL. If there are episodes of hypoglycemia in the premeal period, there may be a reduction of 2.0% from the previous day in basal insulin from the previous day. If there are any episodes of hypoglycemia during the postprandial phase, the dosage of preprandial insulin may be reduced by 2.0% before meals (0.7% per meal in premeal insulin).

In another embodiment of the present invention, provided are methods for treating newly diagnosed and preexisting type 2 diabetes comprising administering one or more agents that stimulate islet neogenesis in combination with one or more beta cell or islet function optimizing agents. The one or more agents that stimulate islet neogenesis include HIP, Optimized HIP, hamster INGAP or other islet neogenesis agents capable of islet regeneration and/transformation of new islets.

The combination of therapies can restore more normal glucose metabolism, including achieving and maintaining appropriate levels of insulin, amylin, glucagon, somatostatin and pancreatic polypeptides, which in turn will improve premeal and postprandial glucose levels, triglycerides, and glucagon and ameliorate the significant weight gain and increased risk for serious hypoglycemia that is associated with tight glycemic control occurring in patients with newly diagnosed and preexisting type 2 diabetes and those with prediabetes and impaired glucose tolerance.

The method is particularly efficacious, because, unlike prior therapies, the therapeutic methods of the invention uniquely promote islet cell regeneration while also optimizing pancreatic islet function of existing islets, while improving other metabolic alterations seen in these conditions. Thus, HIP, Optimized HIP, hamster INGAP and other islet neogenesis agents are working synergistically with other agents that may improve existing pancreatic function. These treatments can be used to improve glycemic control, as measured by hemoglobin AlC, insulin resistance, weight, fluctuations in glucose control, result in decreased premeal and postprandial glucose levels, glucagon, and triglycerides levels, as well as improved lasting glucose, triglycerides, LDL cholesterol, HDL cholesterol and VLDL cholesterol. These methods can also be used to prevent progression of impaired glucose tolerance to diabetes and to prevent progression of impaired fasting glucose to progression, impaired glucose tolerance and prediabetes to the development of diabetes and treat newly diagnosed and preexisting type 2 diabetes.

Embodiments of the present invention also provide methods for treating newly diagnosed or preexisting type 2 diabetes comprising administering an islet cell neogenesis agent in combination with one or more beta cell or islet function optimizing agents. The islet cell neogenesis agents include but not limited to Human proIslet Peptide (HIP), Optimized HIP, hamster INGAP or other agents that result in islet neogenesis. The islet cell neogenesis agent is preferably HIP or Optimized HIP, preferably HIP2 or Optimized HIP2, such as HIP2B. Beta cell or islet function optimizing agents are agents which optimize existing pancreatic islet function, including, but are not limited to: Glucagon Like Peptide-1 (GLP-1) and its analogs, Gastric Inhibitory Peptide/Glucose-Dependent Insulinoptropic polypeptide (GIP), Amylin, and its analog, Pramlintide, and GLP-1 receptor agonists, such as Liraglutide (NN2211) and Exendin-4/exenatide, or compounds which halt the destruction of GLP-1, such as Dipeptidyl Peptidase-4 Inhibitors, (DPP-4 inhibitors), including but not limited to Vildagliptin, Sitagliptin, Saxagliptin, and PHX1149, gastrin, epidermal growth factor-1 and insulin sensitizing agents including the biguanide. Metformin, and the thiazolidinediones, including but not limited to Rosiglitazone and Pioglitazone, AGI-1067, an anti-inflammatory antioxidant agent that works by inhibiting signaling pathways that are activated in response to oxidative stress and pro-inflammatory stimuli, Rimonabant and other drugs that block the cannabinoid receptor 1 (CB1), gut peptide, PYY, inclusive of, but not limited to PYY3-36 (PYY) nasal spray, the hypothalamic neuropeptide Y (NPY) and drugs that impact the leptin, ghrelin, pro-opiomelanocorlin/melanocortin pathways or the melanocortin receptor, orlistat, sibutramine and acarbose. Preferably, the agents are administered in therapeutically effective levels.

In certain embodiments, the method may further comprise administering Vitamin D or a derivative thereof, including, but not limited to cholecalciferol and 1,25 dihydroxyvitamin D.

If a patient is on insulin, mid is diagnosed with type 2 diabetes, confirmation of their type 2 status may be made by evaluating stimulated C-peptide levels to confirm the presence of endogenous insulin along with an autoimmunity assessment to rule out either type 1 diabetes or Latent Autoimmune Diabetes of Adulthood (LADA). If C-peptide levels are significantly low with concomitant immune studies indicative of either type 1 diabetes or LADA, patients may require immune tolerance agent(s) and follow the above protocol for new diagnosed or preexisting type 1 diabetes.

In an insulin-using patient, once the confirmation of type 2 diabetes is made, optimization, of insulin or other injectable diabetes agents, oral diabetic agents and lifestyle modification may be conducted prior to administration of HIP, Optimized HIP, hamster INGAP or other islet neogenesis agents.

Prior to initiation of HIP, Optimized HIP, hamster INGAP or other islet neogenesis agents, there may be a 4-week period in which patients will intensify their glycemic status. Dining this period of optimization of glycemic control, the glucose goal for patients may be between 100 and 200 mg/dL at all times. In order to achieve the optimized glucose goals to initiate HIP, Optimized HIP or other islet neogenesis agents, patients may utilize a medical team with state-of-the-art diabetes tools including subcutaneous continuous monitoring systems. The primary goal be to ensure that no glucose levels falls below 70 mg/dL during the optimization period.

If a patient is on insulin, a sulfonylurea or meglitinide oral agent or other diabetic agent, the sulfonylurea or meglitinide may be changed to one or more of the following agents that optimize beta cell or islet function: Glucagon Like Peptide-1 (GLP-1) and its analogs. Gastric Inhibitory Peptide/Glucose-Dependent Insulinoptropic polypeptide (GIP), Amylin, and its analog, Pramlintide, and GLP-1 receptor agonists, such as Liraglutide (NN2211) and Exendin-4/exenatide, or compounds which halt the destruction of GLP-1, such as Dipeptidyl Peptidase-4 Inhibitors, (DPP-4 inhibitors), including but not limited to Vildagliptin, Sitagliptin, Saxagliptin, and PHX1149. Other compounds which may improve existing islet function include: gastrin, epidermal, growth factor-1 and insulin sensitizing agents including the biguanide, Metformin, and the thiazolidinediones, including but not limited to Rosiglitazone and Pioglitazone, AGI-1067, an anti-inflammatory antioxidant agent that works by inhibiting signaling pathways that are activated in response to oxidative stress and pro-inflammatory stimuli, Rimonabant and other drugs that block the cannabinoid receptor 1 (CB1), gut peptide, PYY, inclusive of, but not limited to PYY3-36 (PYY) nasal spray, the hypothalamic neuropeptide Y (NPY) and drugs that impact the leptin, ghrelin, pro-opiomelanocortin/melanocortin pathways or the melanocortin receptor, orlistat, sibutramine and acarbose.

For example, a patient presenting on 1 mg TID of Repaglinide and 1000 mg of metformin, the Repaglinide may be discontinued and one dosage of 100 mg of Sitagliptin may be initiated instead to maintain postprandial glucose control. If the patient requires more optimal lasting and premeal glucose levels, the metformin may be titrated to 2 grams per day during the four-week optimization of glucose period, prior to the administration of the islet neogenesis agent.

In another example, if a patient has been on 120 mg TID of Nateglinide before meals, this may be discontinued and exenatide/exendin-4 initiated at 5 meg delivered subcutaneously before the two biggest meals. The decisions on which beta cell or islet function optimizing agent may be based upon the glycemic excursions of the individual patients and their response and tolerance to agents such as Glucagon Like Peptide-1 (GLP-1) and its analogs. Gastric Inhibitory Peptide/Glucose-Dependent Insulinoptropic polypeptide (GIP), Amylin, and its analog, Pramlintide, and GLP-1 receptor agonists, such as Liraglutide (NN2211) and Exendin-4/exenatide, or compounds which halt the destruction of GLP-1, such as Dipeptidyl Peptidase-4 Inhibitors, (DPP-4 inhibitors), including but not limited to Vildagliptin, Sitagliptin, Saxagliptin, and PHX1149. Other compounds which may improve existing islet function include: gastrin, epidermal growth factor-1 and insulin sensitizing agents including the biguanide. Metformin, and the thiazolidinediones, including but not limited to Rosiglitazone and Pioglitazone, AGI-1067, an anti-inflammatory antioxidant agent that works by inhibiting signaling pathways that are activated in response to oxidative stress and pro-inflammatory stimuli, Rimonabant and other drugs that block the cannabinoid receptor 1 (CB1), gut peptide, PYY, inclusive of, but not limited to PYY3-36 (PYY) nasal spray, the hypothalamic neuropeptide Y (NPY) and drugs that, impact the leptin, ghrelin, pro-opiomelanocortin/melanocortin pathways or the melanocortin receptor, orlistat, sibutramine and acarbose.

For example, during this optimization of glucose, metformin may also be added or further optimized if a patient is already on metformin. Dosages may be optimized to a total of 2 grams per day in two divided dosages or in one dosage prior to the evening, meal if a one-a-day, sustained preparation of metformin is used.

There may be a two week period prior to the initial administration of HIP, Optimized HIP, hamster INGAP or other islet neogenesis agents, in which the patients should not have any episodes of symptomatic hypoglycemia. Should patients exhibit symptomatic hypoglycemia once HIP, Optimized HIP, hamster INGAP or other islet neogenesis agents is administered, the patients' diabetes regimen may be modified because hypoglycemia may negate the effects of all islet neogenesis compounds including HIP, Optimized HIP, hamster INGAP or other islet neogenesis agents.

HIP, Optimized HIP, hamster INGAP or other islet neogenesis agents may be delivered within 15 minutes of the major meals eaten throughout the day or night, preferably when 30 grams of carbohydrates are consumed. These agents may be delivered via subcutaneous injection, continuous subcutaneous pump delivery or orally. For example, the dosage of HIP and Optimized HIP range from 5-150 mg/kg/day, preferably with each major meal, depending on whether the preparation is oral or subcutaneous and depending upon the selected HIP or Optimized HIP peptide delivered.

Patients initiated on HIP, Optimized HIP, or other islet neogenesis agents who have new onset or preexisting type 2 diabetes, prediabetes, impaired glucose tolerance, are not required to be on a concomitant sulfonylurea or meglitinide in order to reduce the risk for hypoglycemia. An agent that may be more optimal for optimizing beta cell or islet function in existing islets may be initiated instead of a sulfonylurea or meglitinide during the glucose optimization phase prior to administration of the islet neogenesis agent.

During the period in which HIP, Optimized HIP, hamster INGAP or other islet neogenesis agents are administered, the patients' insulin dosage or premeal diabetes medications will be decreased daily as required to prevent any episodes of hypoglycemia and to maintain glucose levels in an optimal range for islet neogenesis. For the first about 30 days, for patients on insulin, there may be a total reduction in insulin as required to prevent any episodes of hypoglycemia and to maintain glucose levels in an optimal range for islet neogenesis and may initially be reduced by 1% per day from the preprandial insulin for those patients on insulin.

During days about 31-60 on HIP, Optimized HIP, hamster INGAP or other islet neogenesis agents, there may be a reduction in insulin among insulin-treated patients to prevent any episodes of hypoglycemia and to maintain glucose levels in an optimal range for islet neogenesis and may initially be reduced by a 1% per day reduction in the basal insulin from the previous day.

During the first about 60 days of islet, neogenesis therapy, patients may have daily communication via phone, e-mail or office visits to give feedback on glucose values to the diabetes health care team. Based on the glucose values, more aggressive reduction in basal insulin dosages may occur if premeal glucose levels are less than 100 mg/dL and more aggressive reductions in premeal insulin may occur if 2 hour postprandial levels are less than 140 mg/dL.

During about days 61-90 on islet neogenesis agent therapy, insulin dosages maybe reduced by 0.5-2.0% per day based upon daily glucose values. Reduction in basal insulin dosages may be required to prevent, any episodes of hypoglycemia and to maintain glucose levels in an optimal range for islet neogenesis and may initially be reduced by 0.5% per day if premeal values are 100-125 mg/dL and a 0.6% per day total reduction (0.18% per meal reduction from the previous day) in premeal insulin may occur if 2 hour postprandial levels are 140-160 mg/dl. Each day there may be a 1% reduction in basal insulin if premeal glucose levels are less than 100 mg/dL and 1% reduction (0.33% per meal reduction from the previous day) in premeal insulin may occur if 2 hour postprandial levels are less than 140 mg/dL. If there are episodes of hypoglycemia in the premeal period, there may be a reduction of 2.0% from the previous day in basal insulin from the previous day if there are any episodes of hypoglycemia during the postprandial phase, the dosage of preprandial insulin may be reduced by 2.0% before meals (0.7% per meal in premeal insulin).

HIP, Optimized HIP, hamster INGAP or other islet neogenesis agents may be delivered within 15 minutes of the major meals eaten throughout the day or night, preferably when 30 grams of carbohydrates are consumed. These agents may be delivered via subcutaneous injection, continuous subcutaneous pump delivery or orally. For example, the dosage of HIP and Optimized HIP range from 5-150 mg/kg/day, preferably with each major meal, depending on whether the preparation is oral or subcutaneous and depending upon the selected HIP or Optimized HIP peptide delivered.

These methods can be practiced to treat a number of diabetes related conditions, including but not limited to newly diagnosed and preexisting type 2 diabetes. These treatments can be used to improve glycemic control, as measured by hemoglobin AlC, insulin resistance, weight, fluctuations in glucose control, result in decreased postprandial glucose, glucagon, and triglycerides levels, as well as improved lasting glucose, triglycerides, LDL cholesterol, HDL cholesterol and VLDL cholesterol. These methods can also be used to prevent progression of impaired glucose tolerance to diabetes and to prevent progression of impaired fasting glucose to progression to impaired glucose tolerance and prediabetes and to treat newly diagnosed type 2 diabetes.

In another embodiment of the present invention, provided are methods for treating conditions relating to aberrant glucose regulation or pathologies associated with impaired pancreatic function comprising administering an islet cell neogenesis agent in combination with one or more beta cell or islet function optimizing agents. Such conditions included, but not limited to, prediabetes, impaired glucose tolerance, insulin resistant syndromes, the metabolic syndrome, obesity, overweight, polycystic ovarian syndrome (PCOS), anovulatory cycles, fasting hyperlipidemia/hypercholesterolemia, elevated fasting total cholesterol, elevated LDL and VLDL cholesterol, family history of diabetes and some forms of impotence and sexual dysfunction associated with the metabolic syndrome, overweight obesity, insulin resistance or inactivity. The islet cell neogenesis agents include but not limited to Human proIslet Peptide (HIP), Optimized HIP, hamster INGAP or other agents that result in islet neogenesis. The islet cell neogenesis agent is preferably HIP or Optimized HIP, preferably HIP2 or Optimized HIP2, such as HIP2B. Beta cell or islet function optimizing agents are agents which optimize existing pancreatic islet function, including, but are not limited to: Glucagon Like Peptide-1 (GLP-1) and its analogs. Gastric Inhibitory Peptide/Glucose-Dependent Insulinoptropic polypeptide (GIP), Amylin, and its analog, Pramlintide, and GLP-1 receptor agonists, such as Liraglutide (NN2211) and Exendin-4/exenatide, or compounds which halt the destruction of GLP-1, such as Dipeptidyl Peptidase-4 Inhibitors, (DPP-4 inhibitors), including but not limited to Vildagliptin, Sitagliptin, Saxagliptin, and PHX1149, gastrin, epidermal growth factor-1 and insulin sensitizing agents including the biguanide, Metformin, and the thiazolidinediones, including but not limited to Rosiglitazone and Pioglitazone, AGI-1067, an anti-inflammatory antioxidant agent that works by inhibiting signaling pathways that are activated in response to oxidative stress and pro-inflammatory stimuli, Rimonabant and other drugs that block the cannabinoid receptor 1 (CB1), gut peptide, PYY, inclusive of, but not limited to PYY3-36 (PYY) nasal spray, the hypothalamic neuropeptide Y (NPY) and drugs that impact the leptin, ghrelin, pro-opiomelanocorlin/melanocortin pathways or the melanocortin receptor, orlistat, sibutramine and acarbose. Preferably, the agents are administered in therapeutically effective levels.

The islet regeneration compound include HIP, Optimized HIP, hamster INGAP or another islet neogenesis compounds, which optimize the production of new pancreatic islets. Such compounds may be used alone or in combination with a single or multiple agents which may optimize pancreatic function of existing islets, improve insulin resistance at the level of the liver or peripheral tissues including muscle and adipose cells, impact the nucleus accumbens receptor in the hypothalamus affecting satiety and impacting food intake and weight, delay gastric emptying creating an earlier feeling of satiety and include, but not limited to Glucagon Like Peptide-1 (GLP-1) and its analogs. Gastric Inhibitory Peptide/Glucose-Dependent Insulinoptropic polypeptide (GIP), Amylin, and its analog, Pramlintide, and GLP-1 receptor agonists, such as Liraglutide (NN2211) and Exendin-4/exenatide, or compounds which halt the destruction of GLP-1, such as Dipeptidyl Peptidase-4 Inhibitors, (DPP-4 inhibitors), and including but not limited to Vildagliptin, Sitagliptin, Saxagliptin, and PHX1149. Other compounds which may improve existing islet function include: gastrin, epidermal growth factor-1 and insulin sensitizing agents including the biguanide, Metformin, and the thiazolidinediones, including but not limited to Rosiglitazone and Pioglitazone, AGI-1067, an anti-inflammatory antioxidant agent that works by inhibiting signaling pathways that are activated in response to oxidative stress and pro-inflammatory stimuli, Rimonabant and other drugs that block the cannabinoid receptor 1 (CB1), gut peptide, PYY, inclusive of, but not limited to PYY3-36 (PYY) nasal spray, the hypothalamic neuropeptide Y (NPY) and drugs that impact the leptin, ghrelin, pro-opiomelanocortin/melanocortin pathways or the melanocortin receptor, orlistat, sibutramine and acarbose.

Because these associated conditions stress islets and are associated with increased and early apoptosis/death of islet and beta cells, HIP, Optimized HIP, hamster INGAP, and islet neogenesis agents are used alone and in combination with one or more of the following agents listed above, which can address the underlying pathophysiology of these conditions and improve or treat such conditions.

Because of the following conditions are pancreatic stressors that result in early beta cell and islet dysfunction and early apoptosis/cell death including elevated fasting glucose levels ≧100 mg/dL, fasting insulin abnormalities, prediabetes, impaired glucose tolerance, insulin resistant syndromes, the metabolic syndrome, obesity, overweight, polycystic ovarian syndrome (PCOS), anovulatory cycles, fasting hyperlipidemia/hypercholesterolemia, elevated fasting total cholesterol, elevated LDL and VLDL cholesterol, and some forms of impotence and sexual dysfunction associated with the metabolic syndrome, overweight obesity, insulin resistance or inactivity, HIP, Optimized HIP, hamster INGAP and other islet neogenesis agents may be utilized to restore islet function and may improve their underlying condition. The beta cell or islet function optimizing agents that may be used include: Glucagon Like Peptide-1 (GLP-1) and its analogs. Gastric Inhibitory Peptide/Glucose-Dependent Insulinoptropic polypeptide (GIP), Amylin, and its analog, Pramlintide, and GLP-1 receptor agonists, such as Liraglutide (NN2211) and Exendin-4/exenatide, or compounds which halt the destruction of GLP-1, such as Dipeptidyl Peptidase-4 Inhibitors, (DPP-4 inhibitors), and including but not limited to Vildagliptin, Sitagliptin, Saxagliptin, and PHX1149. Other compounds which may improve existing islet function include: gastrin, epidermal growth factor-1 and insulin sensitizing agents including the biguanide, Metformin, and the thiazolidinediones, including but not limited to Rosiglitazone and Pioglitazone, AGI-1067, an anti-inflammatory antioxidant agent that works by inhibiting signaling pathways that are activated in response to oxidative stress and pro-inflammatory stimuli, Rimonabant and other drugs that block the cannabinoid receptor 1 (CB1), gut peptide, PYY, inclusive of, but not limited to PYY3-36 (PYY) nasal spray, the hypothalamic neuropeptide Y (NPY) and drugs that impact the leptin, ghrelin, pro-opiomelanocortin/melanocortin pathways or the melanocortin receptor, orlistat, sibutramine and acarbose.

HIP, Optimized HIP, hamster INGAP or other islet neogenesis agents may be delivered within 15 minutes of the major meals eaten throughout the day or night. These agents may be delivered via subcutaneous injection, continuous subcutaneous pump delivery or orally. For example, the dosage of HIP and Optimized HIP range from 5-150 mg/kg/day, preferably with each major meal, depending on whether the preparation is oral or subcutaneous and depending upon the selected HIP or Optimized HIP peptide delivered.

Additionally, based on the patient's underlying medical issue, another agent may selected. For example, if the primary issue is overweight, pramlintide or GLP-1 or a GLP-1 analog may be selected for usage concomitantly with the islet neogenesis agent.

For example, Optimized HIP may be delivered before meals simultaneously with pramlintide delivered within 15 minutes of the major meals in the day or night, preferably when 30 grams, or more of carbohydrates are consumed. Based on whether HIP is delivered subcutaneously or orally, from 5-150 mg/kg/day, preferably divided into doses with each major meal, may be administered along, with pramlintide if the meal contains about 30 grams of carbohydrates or more. Pramlintide is initiated at a dosage of 1.5 micrograms per meal and rapidly titrating up 120, 240 or 360 micrograms subcutaneously before each meal depending on the patient tolerance with tire decision on dosage based up the highest tolerable dosage without significant nausea. The pramlintide dosage may vary if it is delivered in an oral or delivered via a targeted preparation.

Another example is in a patient with PCOS and insulin resistance as manifest by irregular monthly periods and abnormal fasting insulin levels. In addition to initiation of Optimized HIP before meals, sustained release metformin may be initiated at 500 mg per day orally and titrated to 2 grams orally dosed before the evening meal. HIP may be administered prior to each meal along with the metformin, with dinner.

Embodiments of the present invention provide combination therapies and methods for treating newly diagnosed or preexisting type 1, newly diagnosed and preexisting type 2 diabetes mellitus and related conditions such as prediabetes, impaired glucose tolerance, insulin resistant syndromes, the metabolic syndrome, obesity, overweight, polycystic ovarian syndrome (PCOS), anovulatory cycles, fasting hyperlipidemia/hypercholesterolemia, elevated fasting total cholesterol, elevated LDL and VLDL cholesterol, family history of diabetes and some forms of impotence and sexual dysfunction associated with the metabolic syndrome, overweight obesity, insulin resistance or inactivity.

Embodiments of the present invention also provide kits comprising an agent that stimulates islet neogenesis in combination with one or more immune tolerance agents. Further embodiments provide kits further including Vitamin D or a derivative thereof. Further embodiments provide kits further including beta cell or islet function optimizing agents.

Embodiments of the present invention also provide kits comprising an agent that stimulates islet, neogenesis in combination with one or more beta cell or islet function optimizing agents. Further embodiments provide kits further including Vitamin D or a derivative thereof.

Embodiments of the present invention provide a therapeutic composition comprising an agent that stimulates islet neogenesis and an immune tolerance agents.

Embodiments of the present invention provide a therapeutic composition comprising an agent that stimulates islet neogenesis and a beta cell or islet function optimizing agent.

A further embodiment of the present invention provides a method for treating type 1 diabetes mellitus in a patient, said method comprising administering to said patient an agent that stimulates pancreatic islet cell regeneration and/or transformation of new insulin producing islets, which includes, but is not limited to HIP, Optimized HIP, hamster INGAP delivered in a specified protocol in combination with Vitamin D and Vitamin D derivatives, which may include cholecalciferol, 1,25 dihydroxyvitamin D, with an agent or agents that is specifically designed to inhibit the activity of or kill or otherwise cause the death of autoimmune cells, that can cause the death of the pancreatic cells that produce insulin, which may include but not limited to: Mycophenolate mofetil, Daclizumab, Rituximab (anti CD20), anti CD3 antibodies including hOKT3 gamma 1 (Ala-Ala), also known as MGA031 and the monoclonal antibody TRX4 (ChAglyCD3), CTLA4-Ig (Abatacept) a selective costimulation modulator as it inhibits the costimulation of T-cells, Campath-1H, (Anti-CD52 Antibody), a humanized monoclonal antibody to T-cells. Polyclonal Anti-T-Lymphocyte Globulin (ATG), DiaPep277, and anti-GAD antibody vaccine based on the 65 kDa isoform of the recombinant human glutamic acid decarboxylase protein (rhGAD65).

In such a patient, prior to the administration of Human proIslet Peptide (HIP), Optimized HIP, hamster INGAP other islet neogenesis agents, the method may comprise administering to that patient an agent that stimulates pancreatic islet cell regeneration and/or transformation from pancreatic progenitor cells into islet cells with prior initiation of an agent or agents that inhibits the activity of and or block destruction of new islets.

Further embodiments of the present invention provides methods for one or more agents that stimulate or optimize pancreatic islet cell regeneration and/or transformation of new insulin producing cells from to treat conditions often relating to aberrant glucose regulation or pathologies associated with impaired pancreatic function including but not limited to prediabetes, impaired glucose tolerance, insulin resistant syndromes, the metabolic syndrome, obesity, overweight, polycystic ovarian syndrome (PCOS), anovulatory cycles, fasting hyperlipidemia/hypercholesterolemia, elevated fasting total cholesterol, elevated LDL and VLDL cholesterol, family history of diabetes and some forms of impotence and sexual dysfunction associated with the metabolic syndrome, overweight obesity, insulin resistance or inactivity.

Because these conditions are pancreatic stressors that result in early beta cell and islet dysfunction and early apoptosis/cell death with increased and early apoptosis/death of islet and beta cells, HIP, Optimized HIP, hamster INGAP, and islet neogenesis agents are used alone and in combination with one or more of the following agents, which can address the underlying pathophysiology of these conditions and improve or treat such conditions. These agents include: Glucagon Like Peptide-1 (GLP-1) and its analogs. Gastric Inhibitory Peptide/Glucose-Dependent Insulinoptropic polypeptide (GIP), Amylin, and its analog, Pramlintide, and GLP-1 receptor agonists, such as Liraglutide (NN2211) and Exendin-4/exenatide, or compounds which halt the destruction of GLP-1, such as Dipeptidyl Peptidase-4 Inhibitors, (DPP-4 inhibitors), including but not limited to Vildagliptin, Sitagliptin, Saxagliptin, and PHX1149. Other compounds which may improve existing islet function include: gastrin, epidermal growth factor-1 and insulin sensitizing agents including the biguanide, Metformin, (excluded in patients with type 1 diabetes) and the thiazolidinediones, including but not limited to Rosiglitazone and Pioglitazone. Other agents, which may impact pancreatic function, to be utilized with HIP, Optimized HIP, hamster INGAP or other islet neogenesis agents include: AGI-1067, an anti-inflammatory antioxidant agent that works by inhibiting signaling pathways that are activated in response to oxidative stress and pro-inflammatory stimuli, Rimonabant and other drugs that block the cannabinoid receptor 1 (CB1), gut peptide, PYY, inclusive of but not limited to PYY3-36 (PYY) nasal spray, the hypothalamic neuropeptide Y (NPY) and drugs that impact the leptin, ghrelin, pro-opiomelanocortin/melanocortin pathways or the melanocortin receptor, orlistat, sibutramine and acarbose.

Exogenous injectable insulin is a therapy for patients with type 1 diabetes and other conditions in which insulin is either absent or present in diminished or inadequate amounts relative to the glucose content in the bloodstream. Insulin therapy does not treat the underlying mechanisms disease resulting in type 1 diabetes and other such conditions in which there is diminished endogenous insulin production. The therapies, methods, modalities, and treatments described herein are the first to address the many facets of the cause and complications of diabetes. The unique therapies provided by the invention encompass diverse aspects diabetology, metabolism, and immunology. These therapies include those that bring the many different hormones, in addition to insulin, that are diminished or absent in type 1 diabetes. Embodiments of the invention provide for the regeneration, of new insulin producing cells and immuno-modulation that together serve to ameliorate, diminish, or abolish the need for insulin among patients with type 1 diabetes and other conditions associated with inadequate insulin production and secretion.

In type 1 diabetes, there are several, underlying mechanisms that result in significant reduction in the production of insulin. These include autoimmune destruction of the insulin-producing cells and reduction in regeneration capacity of the beta cells with dysfunction of alpha cells and other gluco-regulatory mechanisms. Embodiments of the present invention are efficacious, because even when the autoimmune cells that attack the pancreatic islet cells are blocked at the onset of diagnosis, regeneration of islets is often too slow, even for patients with newly diagnosed type 1 diabetes to become insulin independent. Thus the combination with an islet regeneration peptide will increase efficacy over blocking the immune system.

Embodiments of the invention can even render some patients completely free of their dependence on administered insulin. When new islets are formed, not only are insulin and amylin replaced, which are secreted from the beta cells, but all four cell types within the islet are regenerated. Thus, diminished hormones other than insulin are replaced, and islet regeneration can significantly diminish or abolish insulin requirements in type 1 patients with significantly improved glucose control. By providing new islet cells and blocking the immune cells that can kill them, the methods of the present invention have even greater promise, because they result in the sustained endogenous production of insulin and amylin, resulting in improved, regulation of numerous glucoregulatory feedback mechanisms both with the islet to the alpha cells regulating glucagon and central receptors affecting glucose regulation in the nucleaus accumbens and in the are postrema of the brain.

The present invention provides new methods and pharmaceutical compositions for treating type 1 and type 2 diabetes mellitus and other conditions in which there is a metabolic impairment which may impact pancreatic endocrine function that includes but not limited to insulin secretion, including lasting hyperglycemia, insulin resistant syndrome, hyperglycemic conditions generally in children or adults and those with a family history of diabetes exhibiting an abnormal fasting glucose or insulin levels, metabolic syndrome, being overweight, obesity, polycystic ovarian syndrome (PCOS), anovulatory cycles, fasting hyperlipidemia/hypercholesterolemia, elevated fasting total cholesterol, elevated LDL and VLDL cholesterol, family history of diabetes and some forms of impotence and sexual dysfunction associated with such conditions. The methods and compositions of the invention can treat the underlying pathologic mechanisms of these disease conditions. Thus, the methods of the invention diminish, and in some cases eliminate, the need for insulin administration, to patients formerly in need thereof.

In another, embodiment of the present invention, the method comprises administering an islet neogenesis agent selected from HIP, Optimized HIP, hamster INGAP or other islet neogenesis agents in combination with one or more agents that may optimize existing islet or beta cell function selected from one or more of the following: Glucagon Like Peptide-1 (GLP-1) and its analogs, Gastric Inhibitory Peptide/Glucose-Dependent Insulinoptropic polypeptide (GIP), Amylin, and its analog, Pramlintide, and GLP-1 receptor agonists, such as Liraglutide (NN2211) and Exendin-4/exenatide, or compounds which halt the destruction of GLP-1, such as Dipeptidyl Peptidase-4 Inhibitors, (DPP-4 inhibitors), including but not limited to Vildagliptin, Sitagliptin, Saxagliptin, and PHX1149, gastrin, epidermal growth factor-1 and insulin sensitizing agents including the biguanide, Metformin, (excluded in patients with type 1 diabetes) and the thiazolidinediones, including but not limited to Rosiglitazone and Pioglitazone, AGI-1067, an anti-inflammatory antioxidant agent that works by inhibiting signaling pathways that are activated in response to oxidative stress and pro-inflammatory stimuli, Rimonabant and other drugs that block the cannabinoid receptor 1 (CB1), gut peptide, PYY, inclusive of, but not limited to PYY3-36 (PYY) nasal spray, the hypothalamic neuropeptide Y (NPY) and drugs that impact the leptin, ghrelin, pro-opiomelanocortin/melanocortin pathways or the melanocortin receptor, orlistat, sibutramine and acarbose.

Those of skill hi the art will appreciate in view of the disclosure herein that more than one agent that stimulates islet cell regeneration and/or progenitor cell transformation and/or which slows the degradation of such agents can be used in combination, in the methods of the invention.

In the embodiments of the present invention, the selected agent for increasing islet number, mass, and/or production of endogenously produced insulin is used in combination with a specific agent that inhibits, blocks the activity of or destroys autoimmune cells that target the pancreatic, islet cells. Such immune tolerance agents include, for example, peptides, proteins, and synthetic compounds. In one embodiment, the agent is the anti CD3 antibody, hOKT3 gamma1 (Ala-Ala), also known as MGA031, and the monoclonal antibody TRX4 (ChAglyCD3), Polyclonal Anti-T-Lymphocyte Globulin (ATG), CTLA4-Ig (Abatacept) a selective costimulation modulator as it inhibits the costimulation of T cells, Campath-1H, (Anti-CD5.2Antibody), a humanized monoclonal antibody to T-cells. Polyclonal Anti-T-Lymphocyte Globulin (ATG), DiaPep277, a derivative Heat Shock Protein 60, that may activate a subgroup, of T-cells, which down-regulate T lymphocytes, anti-GAD antibody vaccine based on the 65 kDa isoform of the recombinant human glutamic acid decarboxylase protein (rhGAD65) and other compounds that specifically delay, prevent, or halt autoimmune destruction of the islet cell. Those of skill in the art will appreciate in view of the disclosure herein that more than one agent that blocks autoimmune destruction of pancreatic islet cells can be used in combination in the methods of the invention.

Thus, embodiments of the combination therapies and related methods involve the co-administration of one or more agents that stimulate islet cell regeneration or progenitor cell transformation with one or more agents that block autoimmune destruction of pancreatic islet cells. As used herein, an agent is “co-administered” or “used in combination” with another agent (also referred to herein as, “agent”) when the two agents are administered as part of the same course of therapy, in one embodiment, a first agent is first administered prior to administration of the second agent, and treatment with both is continued throughout the course of therapy. In another embodiment, the second agent is administered after the initiation or completion of the therapy involving the first agent. In other embodiments, the first agent is administered contemporaneously with the initiation, of the therapy with the second agent in one embodiment, a therapy involving one or more agents to block or kill autoimmune cells that, target pancreatic islet cells is first administered prior to administration of the therapy that stimulates islet cell regeneration or progenitor cell transformation or both. In one embodiment, treatment with the specific autoimmune blocker is continued after the cessation of treatment with agents that stimulate islet cell, regeneration.

Embodiments of the methods the present invention can involve multiple rounds, or “cycles” of treatment. Each cycle of one or more administrations of an agent that stimulates islet cell regeneration or progenitor cell transformation and one or more administrations of an agent that blocks autoimmune cells that target pancreatic islet cells (as well as a complete set of cycles) can be viewed as practice of the method. Thus, an islet cell regeneration agent can be administered in a subset of such cycles, for example. Those of skill in the art will also appreciate, that in many cases the schedule of co-administration may differ in the first or a later therapeutic cycle for the convenience of the patient.

Embodiments of the combination therapies and related methods of the invention uniquely target the underlying pathologic mechanisms of type 1 diabetes by administering agents that regenerate new islet, cells and/or transform pancreatic progenitor cells into islets in combination with agents that provide immune therapy targeted at protecting new islets generated by the islet neogenesis agents. This combination therapy treats the underlying mechanisms of type 1 diabetes, which is an autoimmune phenomena in which anti-self antibodies attack the pancreas.

Embodiments of the present invention treat the underlying pathologic mechanisms of type 1 diabetes, type 2 diabetes and conditions resulting from decreased insulin production due to an imbalance between destruction, regeneration, and sustenance of insulin producing islet cells. The methods and compounds of the invention can reduce the insulin requirements of patients currently taking the drug due to having type 1 or type 2 diabetes or another disease or condition of impaired glucose metabolism and/or insulin resistance creating abnormal physiology. Embodiments of the present invention can improve glucose control in such patients. In some patients, treatment in accordance with the methods of the invention can ameliorate or obviate the need for administered insulin.

Embodiments of the present invention can be used to treat any mammal, including humans and animals, suffering from a disease, symptom, or condition related to a diminished production of insulin due to the loss of pancreatic islet cells. Such diseases and conditions include, of course, type 1 diabetes mellitus, pre-type 1 diabetes, including but not limited to pre-diabetes in a type 1 patient as manifested by antibodies (anti-GAD65 and others) specific for type 1 diabetes, and latent autoimmune diabetes of adulthood (LADA). Moreover, the present invention can be practiced with therapeutic benefit for patients newly diagnosed as having type 1 diabetes, the siblings and first degree relatives of patients with type 1 diabetes, and people with positive antibodies indicative of future development of type 1 diabetes and other autoimmune conditions that indicate a predilection to type 1 diabetes.

The combination therapies and related methods and compositions can also be employed as adjunctive therapy to insulin therapy in type 1 diabetes in children and adults, to ameliorate glucose swings among patients with diabetes, and in patients with poorly controlled diabetes, hypoglycemic unawareness, and recurrent hypoglycemia in type 1 diabetes.

The therapies and related methods and compositions can be used to treat patients having newly diagnosed or preexisting type 2 diabetes, type 2 diabetes' in children, type 2 diabetes being concurrently treated with insulin therapy, or injectable diabetic agents or oral antidiabetic agents or those treated without medication but with diet, exercise and lifestyle modification with poorly controlled type 2 diabetes, as manifested by an elevated hemoglobin AlC. The methods and compositions of the invention can also be used to treat both children and adults having atypical forms of diabetes and patients having the conditions of fasting or postprandial hyperglycemia.

The therapies and related methods and compositions can also be used to treat patients who are children as well as adult patients in need of weight loss, including but not limited to achieve weight loss or treat obesity in patients having type 1 diabetes as well as those who do not have type 1 or 2 diabetes. In one embodiment, the methods and compositions of the invention are used to treat a patient having obesity or are overweight.

The therapies and related methods and compositions can also be used to children and adults having prediabetes, dysmetabolic syndrome or metabolic syndrome, as well as patients exhibiting the conditions of hyperlipidemia and hypercholesterolemia, fasting LDL or VLDL cholesterol or lasting or postprandial hypertriglyceridemia with and without diabetes and those with a family history of diabetes exhibiting an abnormal fasting glucose or insulin level.

Other patients that can benefit from the therapies and related methods of the invention include children and adult patients diagnosed as having conditions such as fasting hyperglycemia, prediabetes, impaired fasting glucose, impaired glucose tolerance, and hyperglycemic conditions generally in children or adults and those with a family history of diabetes exhibiting an abnormal lasting glucose or insulin level.

The therapies and related methods and compositions of the invention can also be used to treat patients having polycystic ovarian syndrome (PCOS), anovulatory cycles, fasting hyperlipidemia/hypercholesterolemia, elevated fasting total cholesterol, elevated LDL and VLDL cholesterol, family history of diabetes and some forms of impotence and sexual dysfunction associated with the metabolic syndrome, overweight obesity, insulin resistance or inactivity.

The therapies and related methods and compositions of the invention can also be used to treat patients having recurrent pancreatitis or pancreatic cancer and can be used in all modalities of a need for auto islet regeneration/regeneration of one's own islets.

In embodiments of the invention, the agent that stimulates islet, cell regeneration and/or transformation into insulin producing islet cells is used in conjunction with one or more agents which optimize existing islet or beta cell function and include but are not limited to: Glucagon Like Peptide-1 (GLP-1) and its analogs, Gastric Inhibitory Peptide/Glucose-Dependent Insulinoptropic polypeptide (GIP), Amylin, and its analog, Pramlintide, and GLP-1 receptor agonists, such as Liraglutide (NN2211) and Exendin-4/exenatide, or compounds which halt the destruction of GLP-1, such as Dipeptidyl Peptidase-4 Inhibitors, (DPP-4 inhibitors), including but not limited to Vildagliptin, Sitagliptin, Saxagliptin, and PHX1149, gastrin, epidermal growth factor-1 and insulin sensitizing agents including the biguanide, Metformin, (excluded in type 1 patients) and the thiazolidinediones, including but not limited to Rosiglitazone and Pioglitazone, AGI-1067, an anti-inflammatory antioxidant agent that works by inhibiting signaling pathways that are activated in response to oxidative stress and pro-inflammatory stimuli, Rimonabant and other drugs that block the cannabinoid receptor 1 (CB1), gut peptide, PYY, inclusive of, but not limited to PYY3-36 (PYY) nasal spray, the hypothalamic neuropeptide Y (NPY) and drugs that impact the leptin, ghrelin, pro-opiomelanocorlin/melanocortin pathways or the melanocortin receptor, orlistat, sibutramine and acarbose.

These agents are useful in embodiments of the present invention, in that they can be used to optimize existing islet or beta cell function and may work synergistically with HIP, Optimized HIP, hamster INGAP or another islet neogenesis agent to improve glycemic control, as measured by hemoglobin AlC, in diabetes; to prevent progression of impaired glucose tolerance or prediabetes to diabetes, to treat newly diagnosed type 2 diabetes; to treat type 2 diabetes, prediabetes, impaired glucose tolerance, insulin resistant syndromes, the metabolic syndrome, obesity, overweight, polycystic ovarian syndrome (PCOS), anovulatory cycles, fasting hyperlipidemia/hypercholesterolemia, elevated fasting total cholesterol, elevated LDL and VLDL cholesterol, family history of diabetes and some forms of impotence and sexual dysfunction associated with the metabolic syndrome, overweight obesity, insulin resistance or inactivity. Methods, agents, and pharmaceutical formulations useful in the practice of the present invention to achieve pancreatic islet cell regeneration include those described in the following references, each of which is incorporated herein by reference: Rosenberg et al., 1992, Adv. Exp. Med. Biol. 321:95-104; March 1996, Diabetologia 39(3):256-62; July 1996, Pancreas 13(1):38-46; and November 2004, Ann. Surg. 240(5):875-84; Vinik et al., June 1997, Horm. Metab. Res, 29(6):278-93.

Exendin-4 or synthetic exendin-4 is administered at a dose ranging from 5 to 20 micrograms before meals. This dose will provide patients the ability to reduce bolus insulin before meals by 10-20% with reduced fluctuations and decreased postprandial, glucose, glucagon, and triglycerides. Administration of exendin-4 in accordance with the methods of the invention can be used to improve glycemic control, as measured by hemoglobin AlC, in type 1 diabetes; to prevent progression of impaired glucose tolerance in diabetes; to prevent progression of impaired fasting glucose to impaired glucose tolerance and diabetes; to treat newly diagnosed type 2 diabetes; and to treat type 2 diabetes

In one embodiment, HIP or hamster INGAP is administered either orally or subcutaneously or in combination with another islet stimulating peptide or compound and dosed at 5.0-15.0 milligrams per kilogram patient weight per body weight per day subcutaneously. In one embodiment INGAP is administered in a continuous subcutaneous infusion over 24 hours, in one embodiment, INGAP is administered in divided dosages per day before meals when 30 grams or more of carbohydrates are consumed. In one embodiment, INGAP is administered using a continuous infusion device, transdermal patch, microneedle delivery system to provide a consistent basal level delivery of INGAP. In another embodiment, INGAP is delivered in a continuous infusion with bolus delivery before meals. In another embodiment, a sustained release formulation requiring administration no more frequently than once every week, once every 2 weeks, or once monthly injections is employed. In other embodiments, HIP and hamster INGAP may also be delivered with dosages in the range of about 50 to about 150 mg/kg/day, preferably delivered orally in divided dosages before meals.

In another embodiment. Optimized HIP is delivered in a concentration about 5 to about 15 mg/kg/day, more preferably in divided subcutaneous injections in humans. Thus a 60 kg individual would potentially receive 900 mg/day divided into three, 300 mg dosages delivered before meals. In other embodiments, Optimized HIP may also be delivered with dosages in the range of about 50 to about 150 mg/kg/day, preferably delivered orally in divided dosages before meals.

The immune tolerance agents and the beta cell or islet function optimizing agents useful in the methods of the invention can be formulated, administered, and dosed as known in the art or as described herein.

Dosing and administration of the agents useful in the methods of the invention as described herein provide accelerated islet cell regeneration and/or transformation of progenitor cells to optimize an individual's ability to secrete insulin from endogenous, newly formed islet cells as well as the lowest toxicity for the agents that delay or prevent the destruction of pancreatic islet cells. Pharmaceutical compositions of the invention provide for kinetic delivery of these agents, ease of delivery, and enhanced efficacy.

The agents useful in the methods of the invention can be administered by a variety of routes. Known agents useful in the methods of the invention can be administered by routes and using pharmaceutical formulations previously developed for other indications. Such delivery routes include, at least for most known agents, oral delivery, topical delivery, including micelle and nanosphere topical delivery systems, subcutaneous delivery including pump-assisted continuous infusion and disposable micro-pumps and micro-needles (including but not limited to those available from Johnson & Johnson, debiotech and others), and buccal delivery.

Of course, the particular route of administration and pharmaceutical formulation of an agent used in the practice of the methods of the invention will be selected by the practitioner based on a patient's disease or condition being, treated and the agent employed. A wide variety of pharmaceutical compositions can be employed in the methods of the invention. In some embodiments, extended use preparations can be used for ease of administration and increased efficacy. In one embodiment, one or more of the agents employed. In the method is formulated as a micelle.

Often, ease of administration is best achieved by oral delivery. While small molecule pharmaceutical agents can often be readily formulated for oral delivery, peptide and protein-based pharmaceutical agents can be more difficult to formulate for oral delivery. However, suitable formulation technology exists, and in one important aspect, the present invention provides pharmaceutical compositions of proteins and peptides formulated for oral delivery. In one embodiment, the pharmaceutical compositions useful in the methods of the invention suitable for oral delivery are formulated generally in accordance with known Technosphere technology developed by MannKind Corp., Eligen Technology developed by Emisphere, and nasal delivery systems developed by Nastech.

With the foregoing detailed description of the reagents and methods of the invention, the following Examples are provided to illustrate various aspects of the invention.

### EXAMPLE 1

In Vitro Proof of Concept of HIP Activity. The in vitro proof of concept studies were conducted at the University of Pennsylvania Human Islet Laboratory. Human pancreatic islet and ductal fractions were cultured over 10 days and then treated in a blinded study. Radioimmunoassay methods were used to measure insulin levels in the human pancreatic cultures treated with a scrambled peptide serving as a negative control, HIP3, HIP1, HIP2 and hamster-derived INGAP serving as a positive control. Peptides were synthesized by Sachem Bioscience (95% pure, research grade).

Duplicate cultures were treated in both ductal and islet tractions of human pancreatic tissue on day 10 and day 12 and then lysed for detection of insulin content after 1 week of treatment HIP peptides, control and INGAP. During 10-day culture, the insulin production goes down and then after treatment with HIP peptides, insulin is produced again.

The ductal and islet tissue were separated using the Ricordi method. Neither ductal cell nor islet culture was completely homogeneous in nature. The studies also suggest that progenitor cells, which are the target for HIP, are found both in islet and ductal cultures. The studies were repeated with similar findings shown in the following chart, with as much as a four-fold increase in insulin levels by radioimmunoassay among human ductal tissue cultured with HIP 2.

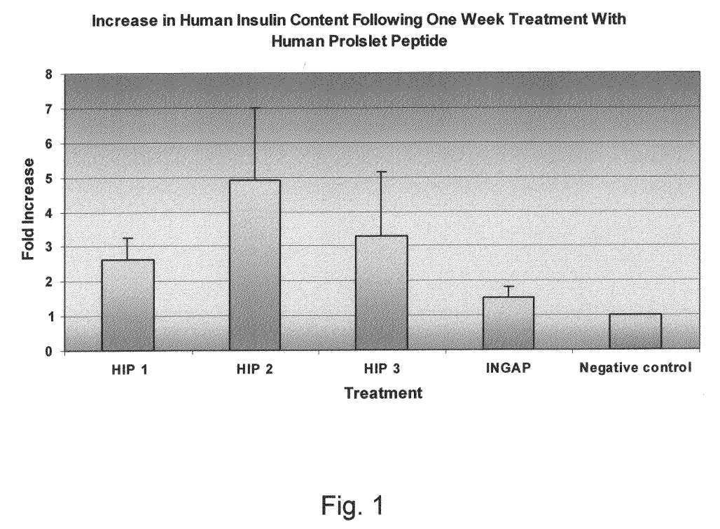

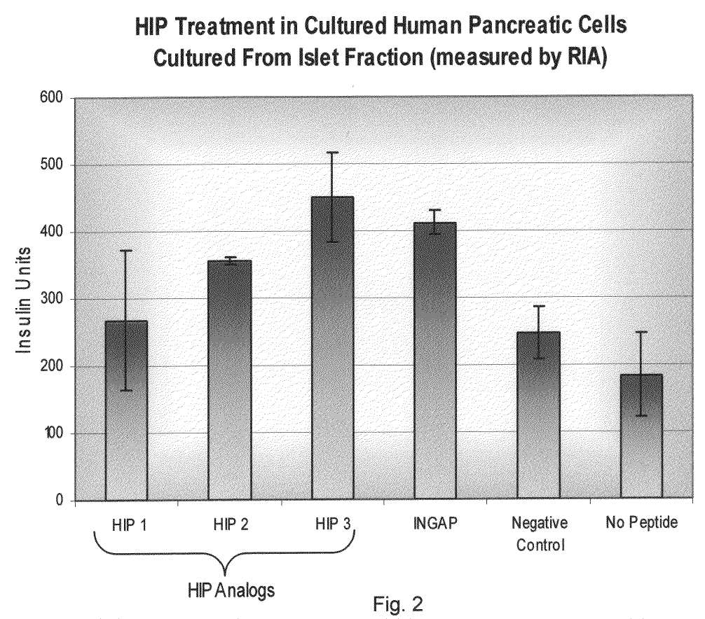

The ductal fraction graph as shown in FIG. 1 depicts the insulin levels on the y axis as measured by radioimmunoassay after incubation in culture with human pancreatic ductal tissue. The islet fraction graph indicates insulin levels after incubation in human pancreatic islet tissue. Baseline insulin levels are significantly higher in the islet fraction at baseline than in the ductal fractions at baseline. Similar studies were conducted with HIP and hamster INGAP in islet fractions, as shown in FIG. 2.

Repeated studies confirmed the increase in insulin both in predominately human ductal cell cultures and islet cultures, with baseline insulin levels consistently about ⅓ lower in the baseline ductal cultures compared to islet cultures, with similar rises in insulin content after incubation with HIP peptides compared to a negative control.

### EXAMPLE 2

In Vivo Studies. HIP3, HIP1, HIP2 and hamster INGAP have been the subject of in vivo studies in mice. Studies have shown that these HIP variants, when introduced into diabetic mice, stimulate differentiation of progenitor cells within the pancreas into new islet structures A model of diabetes has been developed in the mouse (Rosenberg et, al., 2004). The subject number was selected to yield a sufficient number of diabetic animals for the study and animals were randomly assigned to study groups. All animals were dosed via intraperitoneal injections twice daily (am and pm) for 28 consecutive days. The timing of dose administration remained consistent (±2 hours) during the dosing phase. After confirmation that the mice had been diabetic (blood glucose greater than 16.7 mmol/L (300 mg/dL) for at least 1 week, mice were dosed.

Mice were injected intraperitoneally with streptozocin at 40 mg/kg in citrate buffer, pH 4.5, on 5 consecutive days in an attempt to render them diabetic. Mice must have had blood glucose greater than 16.7 mmol/L (300 mg/dL) for at least 1 week to be considered diabetic. If the blood glucose level in any animal rose to above 400 mg/dL, the animal was treated with insulin. Every 3 days, at the same time each day, a nick was made on the tail and a drop of blood was collected. Glucose measurements were determined using a glucose meter. Group assignments and dose levels were as follows in Table 1:

TABLE 1

| | | | | Number of |
|---|---|---|---|---|
| Group | Treatment | Dose Level | Dose Volume | Animals |
| 1 | Vehicle | 0 | 100 μl | 6 |
| 2 | HIP3 | 250 μg | 100 μl | 6 |
| 3 | HIP1 | 250 μg | 100 μl | 6 |
| 4 | HIP2 | 250 μg | 100 μl | 6 |
| 5 | Hamster | 250 μg | 100 μl | 6 |
| | INGAP | | | |

Study endpoints included the following; changes in glucose; changes in insulin requirements; and histology of post-mortem pancreata.

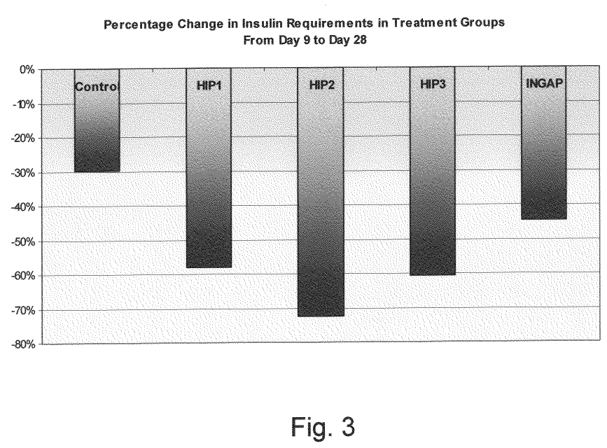

Changes in Insulin Requirements. Reductions in both the insulin requirements and the rate of decrease in insulin requirements were seen among HIP-treated mice and hamster INGAP and placebo-treated groups. FIG. 3 demonstrates the reduction in insulin dosages with HIP 2-treated mice being completely insulin-free by day 21.

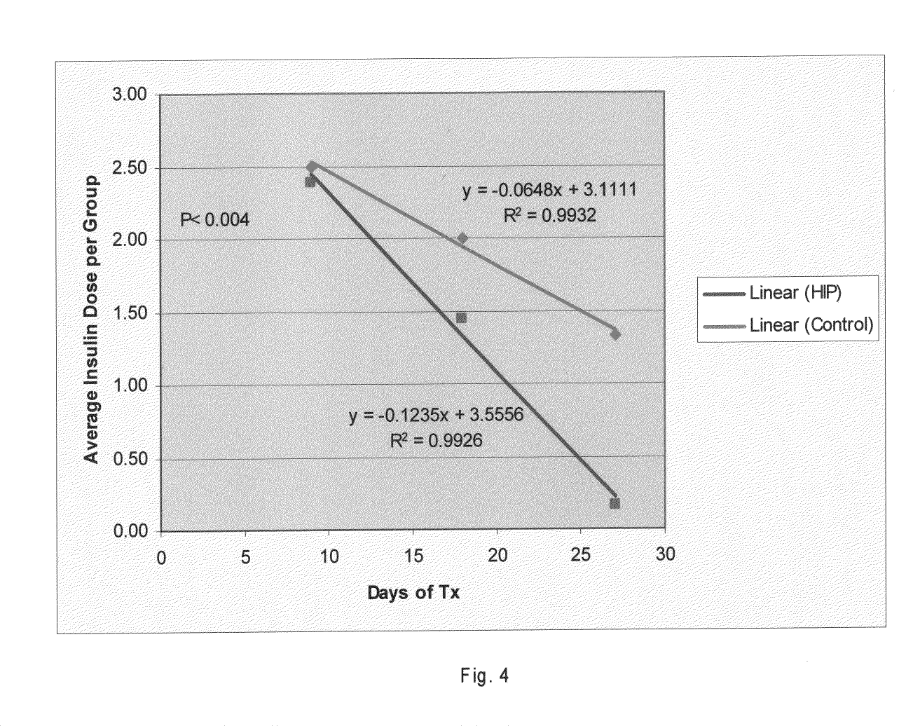

FIG. 4 demonstrates the rate of reduction of insulin required by mice was also significantly faster in HIP-treated diabetic mice compared to control (p=0.004). Based upon glucose levels in mice, insulin was administered, and there were concomitant reductions changes in glucose levels, which are reflected in the reduced need for insulin. There was a 14.7% lower mean glucose between HIP 1 and control, a 29.4% lower mean glucose between HIP 2 and control, and a 57.3% mean lower glucose between HIP3 and the control group. The data indicates the significantly faster rate of decline in insulin requirements among all HIP-treated mouse groups compared to control diabetic mice. There were significantly greater numbers of islets after HIP treatment observed in mouse pancreata, which were sectioned and reviewed on each mouse studied. The pancreata were evaluated by a histologist blinded the specimens with the following data shown in Table 2,

TABLE 2

| | Total Islets | Total Islet Mass |
|---|---|---|
| | (% increase) | (μm2) (% increase) |
| Placebo | 280 | 854364 |
| HIP2 | 454 (62%) | 2161782 (153%) |
| HIP3 | 410 (46%) | 1703513 (99%) |

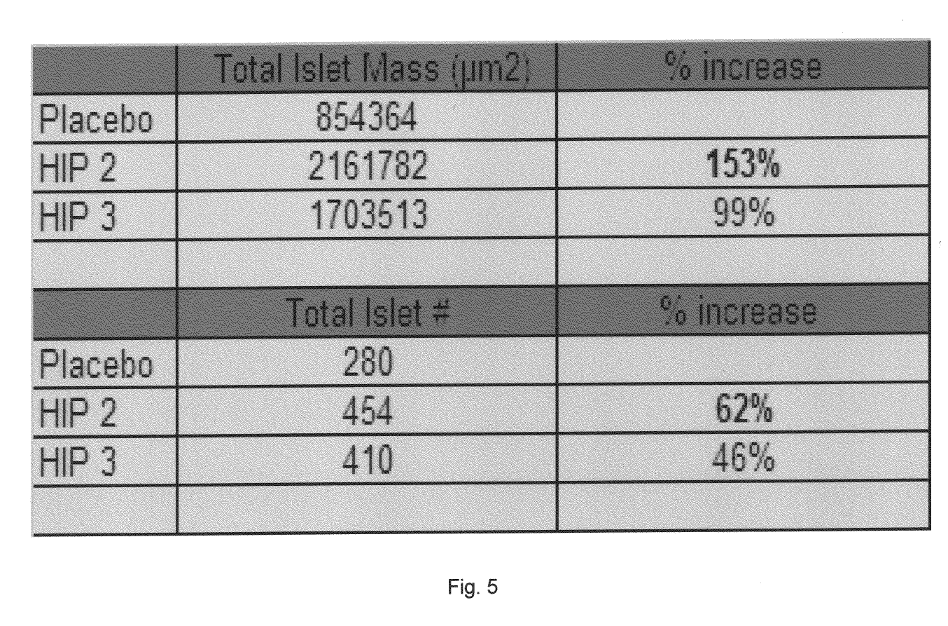

The difference in islet number between HIP and placebo was statistically significant (p=0.022). There was even a more profound increase in islet area between the HIP-treated mice and the placebo-treated group. The islet area in the HIP2-treated group was 360,297 μm(2) compared to 142,394 μm(2) in the placebo-treated group with 283,918 μm(2) in the HIP3-treated group (p=0.05), as shown in FIG. 5.

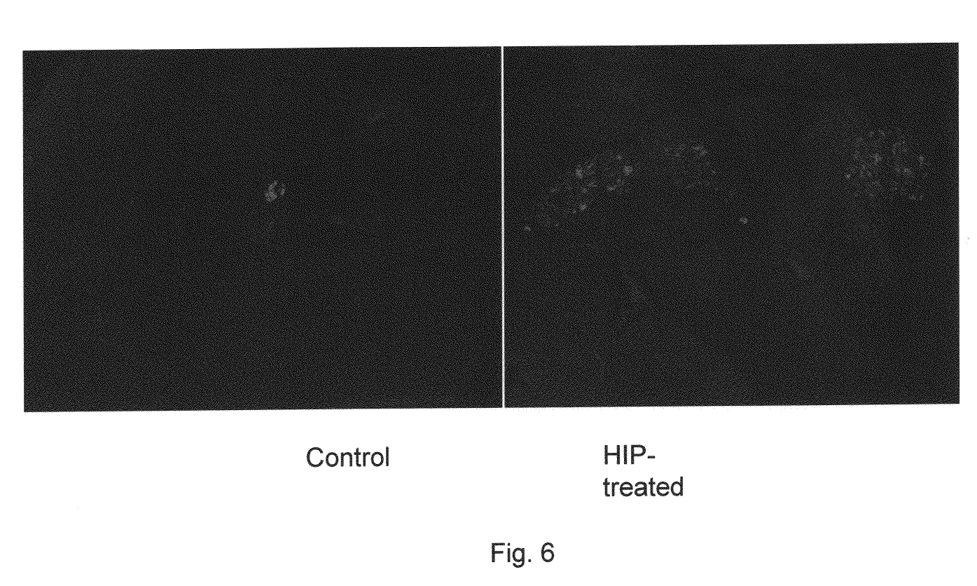

Immunofluorescent staining for insulin was also performed on mouse pancreata demonstrate are greater degree of insulin staining in the HIP-treated mice, as shown in FIG. 6. This mouse pancreas tissue was harvested and fixed in 4% PFA, blocked and sectioned. 10×-Objective, 1.6 optivar.

### EXAMPLE 3

Utilizing a technique of growing an established immortalized human cell line from human pancreatic epitheloid cells known as PANC-1, the impact in vitro of impact of HIPs and Optimized HIPs on insulin production was evaluated. This cell line demonstrates the ability to differentiate into other pancreatic cell types upon appropriate signaling. Therefore, PANC-1 cells were used as a surrogate to the naturally occurring progenitor cells of the pancreas.

PANC-1 cells were seeded in 175 flasks in DMEM media containing 10% fetal bovine serum. The cells were incubated at 37° C., 5% CO2 for 24 hours and then treated with HIPs at the final concentration of 167 nM. This treatment was performed once a day for four days. On the fifth day the cells were broken to obtain the cell lysates. In these cell extracts the total protein levels were determined, and 50 micrograms of total protein were used to perform the western blot analysis. The samples containing 50 micrograms of proteins were diluted in loading buffer containing or not 5% of the reducing agent beta-mercaptoethanol, and loaded into each well of the gel. After the electrophoresis and transfer of the proteins to nitrocellulose membranes, the presence of insulin was detected by using as a primary antibody the polyclonal chicken anti insulin antibody (ab14042, dilution of 1/2000), and as secondary antibody the rabbit polyclonal-HRP conjugated anti-chicken (dilution 1/1000 for the NIT gel and 1/2000 for the PANC-1 gel).

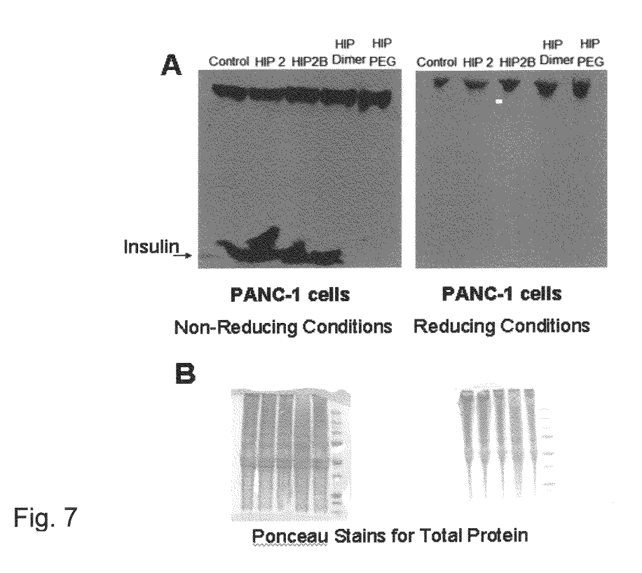

FIG. 7A is a Western Blot analysis demonstrating expression of human insulin from PANC-1 cells in response to incubation with HIP and Optimized HIP under non-reducing and reducing conditions. The panel labeled A demonstrates bands for insulin in the PANC-1 cells when the samples were loaded in non-reducing conditions. FIG. 7B are Ponceau Stains under non-reducing and reducing conditions in response to incubation with various HIP and Optimized HIPs.

FIG. 7B show the total protein contained in the same membranes as in FIG. 7A. The determination of the levels of total protein via Ponceau staining demonstrates that the different lanes contain similar amount of proteins. The total protein levels in the NIT-1 and PANC-1 cells were determined, and 50 micrograms of total protein were used to perform the Western Blot analysis. The samples containing 50 micrograms of proteins were diluted in loading buffer containing or not 5% of the reducing agent beta-mercaptoethanol, and loaded into each well of the gel.

The Ponceau staining demonstrates that the differences in insulin expression respond to different HIPs and Optimized HIPs are not related with amount of protein loaded in the wells. Also the lack of signal for insulin, for example, the membrane in the reducing conditions, is not attributed to a lack of protein.

### EXAMPLE 4

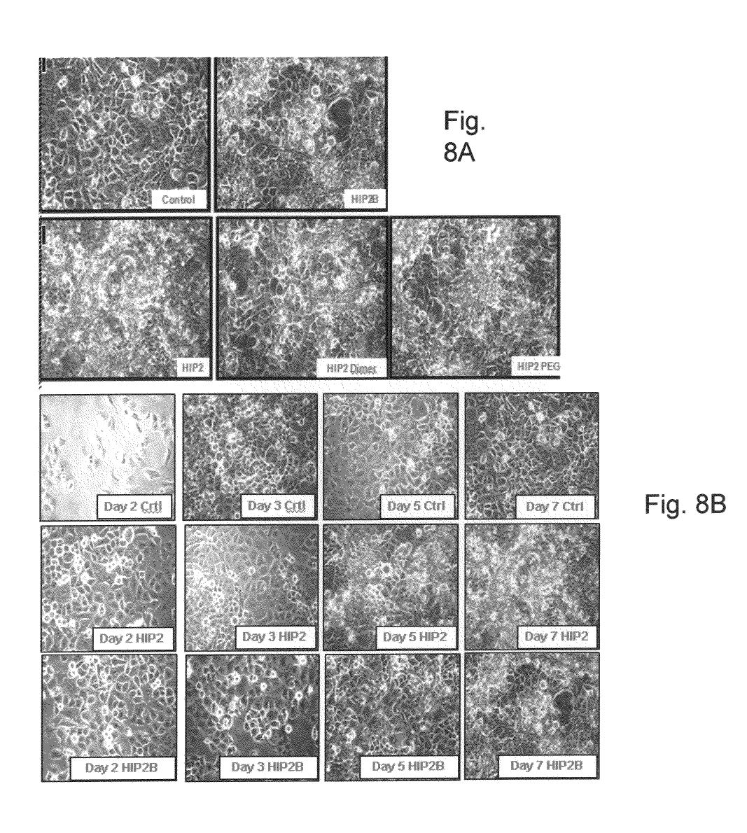

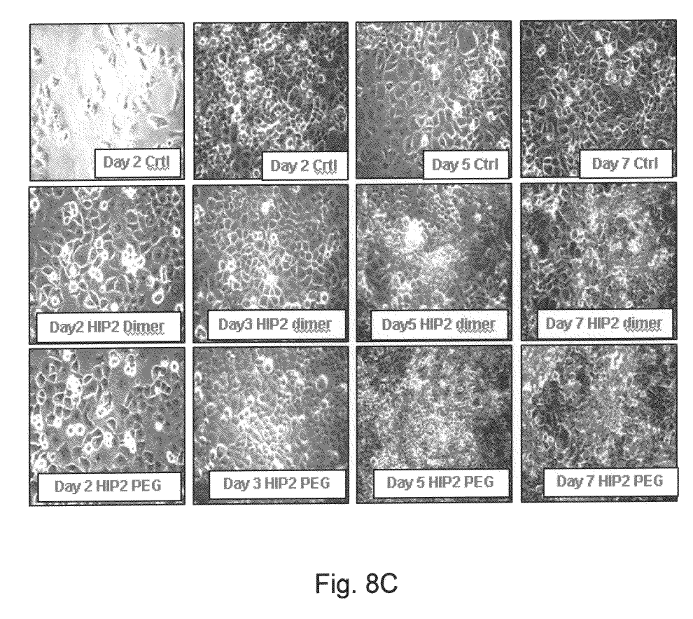

FIG. 8 demonstrates the impact of HIP and Optimized HIP Peptides on Cell Morphology in human PANC-1 Cell Lines. The cells were treated with HIP and Optimized HIP peptides for four days. In FIG. 8A, taken on day 7 at 200× magnification, morphological differences can be seen between, the control condition and the cells treated with HIPs and Optimized HIPS with histologically more differentiated cells, particularly in the HIP2B-treated cells. FIG. 8B show the progression of the cell morphology changes through 7 days, with the control on the top, HIP2 in the middle, and HIP2B on the bottom. Pictures were taken on days 1, 2, 3, 5, and 7 at 200× magnification. While the control-treated cells did not appear to undergo any changes, the cells treated with HIP2 and HIP2B deviate significantly from their initial appearance. FIG. 5C demonstrates the progression of morphological changes when HIP2 Dimer and HIP2 PEG are treated in PANC-1 cell cultures. Overall, the control-treated cells did not undergo any significant visual changes, the cells treated with HIP2 and HIP2B deviate significantly from their initial appearance.

### EXAMPLE 5

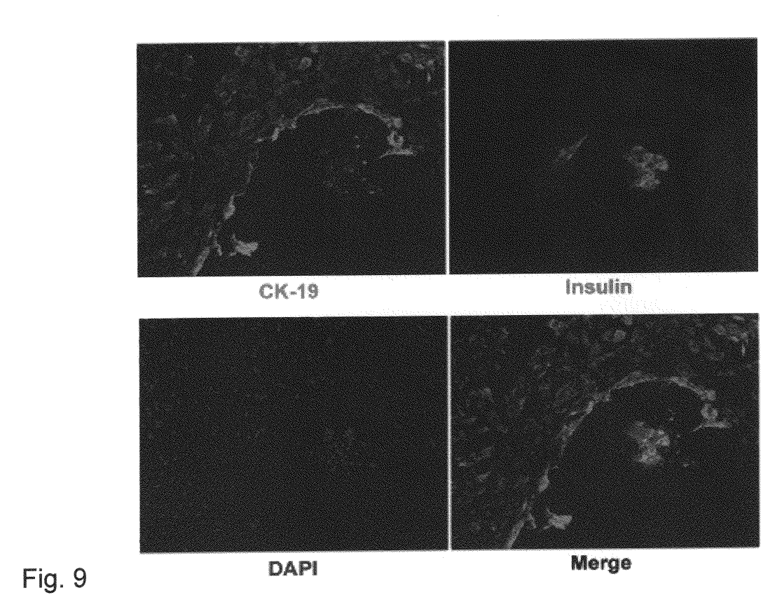

FIG. 9 demonstrates HIP 2B Activity in Human Pancreatic Tissue Culture. Studies demonstrated the impact of HIP and Optimized HIP peptides in, human pancreatic ductal cell cultures. The ductal fraction of human pancreatic cells were cultured for 10 days in a collagen matrix and then treated every other day with HIP2B. Cells were labeled by double antibody staining for CK19, a marker for ductal tissue, and DAP1 staining to show nuclei and insulin. As shown in FIG. 8, the cells underwent morphological changes that induced insulin expression in otherwise insulin negative cells.

### EXAMPLE 6

### Pilot Data of Impact of HIP and Optimized Hip Peptides in the Non Obese Diabetic Model

Consistent with the data in the STZ-treated mice (above) of increased islet mass, area and number, the pilot NOD mouse model demonstrated preliminary evidence of the potential for Optimized HIP to provide better efficacy in terms of islet neogenesis as measured by C-peptide levels in mice following HIP treatment for 39 days.

The non obese diabetic (NOD) model is used as a model for type 1 autoimmune diabetes. This form of diabetes is the most challenging in that the underlying damage to foe pancreas and it's insulin production is due to autoimmune attack. Therefore, in order to show definitive islet neogenesis in this form of diabetes an immune tolerance agent must be used in combination with HIP. The NOD mouse model is extremely difficult model, because many of the mice may only transiently become diabetic and go into remission, whereas others develop severe diabetes. The timing intervention in this transgenic mouse model is difficult to determine.

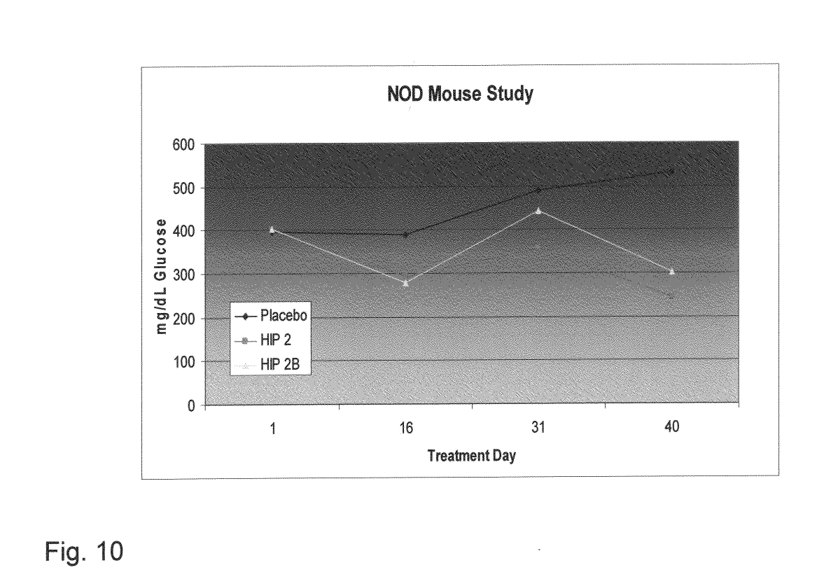

In a preliminary study that utilizing the immune tolerance agent, lysophylline (LSF) under development, three NOD mice that became diabetic and were randomized to placebo plus LSF, HIP2 plus LSF and HIP2B plus LSF. As shown in FIG. 10, of the group who received LSF at the appropriate time, the two treated with HIP responded with steadily improved glucose levels during the study compared to the NOD mouse treated with LSF alone that had gradual elevations in glucose throughout the study. While not a statistically significant study, these data provide very compelling evidence for pursuit of the combination of an immune tolerance agent and HIP for type 1 diabetes.

### EXAMPLE 7

Impact of HIP2B and HIP2 on HIP Receptor. The following sets of studies demonstrate that HIP2B is as effective as HIP2 in the interaction with the cytoplasmic membrane receptor for HIP and trafficking from the receptor to the nucleus. The receptor for Human ProIslet Peptide was labeled using a double antibody method in a stable human pancreatic cell line. The first antibody was a rabbit polyclonal and the second was a goat-anti-rabbit labeled with Cy3 fluorescent dye.

These cells grow normally in serum tree media and when treated with trypsin, are destabilized and made competent to undergo developmental changes. Cells were cultured in serum free media (SFM) with and without HIP, and in serum free media with trypsin (TSFM). This is to show that simply destabilizing, does not activate developmental changes.

When treated with HIP under stable conditions, no changes result. When treated with HIP under developmentally competent conditions, the labeled receptor responds to the presence of HIP by being encapsulated by the cytoplasmic membrane and moving to the nuclear membrane where the signals for differentiation are received.

When treated with HIP under stable conditions, no changes result. When treated with HIP under developmentally competent conditions, the labeled receptor responds to the presence of HIP by being encapsulated by the cytoplasmic membrane and moving to the nuclear membrane where the signals for differentiation are received.

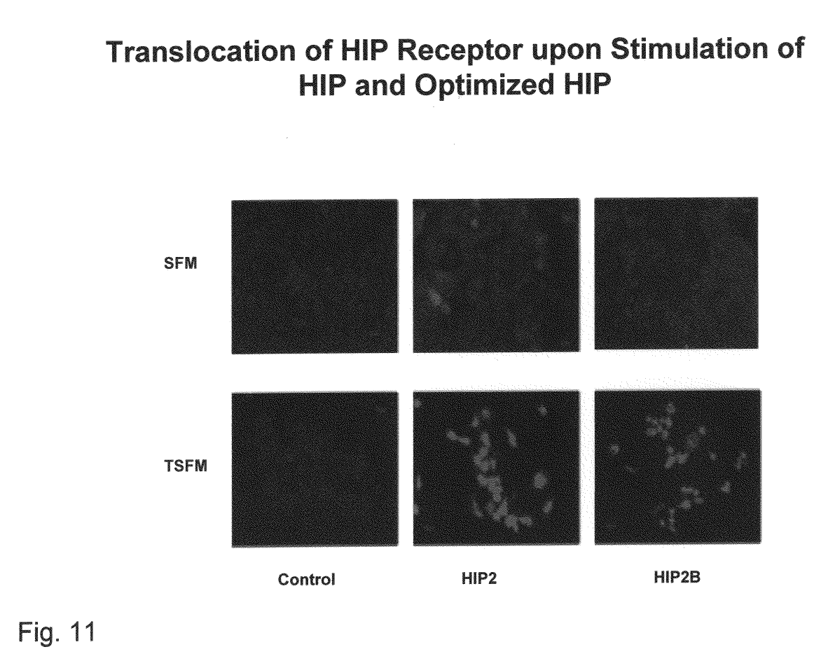

FIG. 11 demonstrates PANC-1 cells treated with trypsin and incubated in serum free medium differentiate into islet cell aggregates. HIP receptor is up-regulated during differentiation of human pancreatic cells mid appears to interact with HIP2 and HIP2B interact with HIP receptor. HIP2 and HIP2B stimulated traffic from the HIP receptor on the cytoplasmic membrane into the nucleus of the cells stimulating differentiation of pancreatic progenitor cells into insulin-producing new islets.

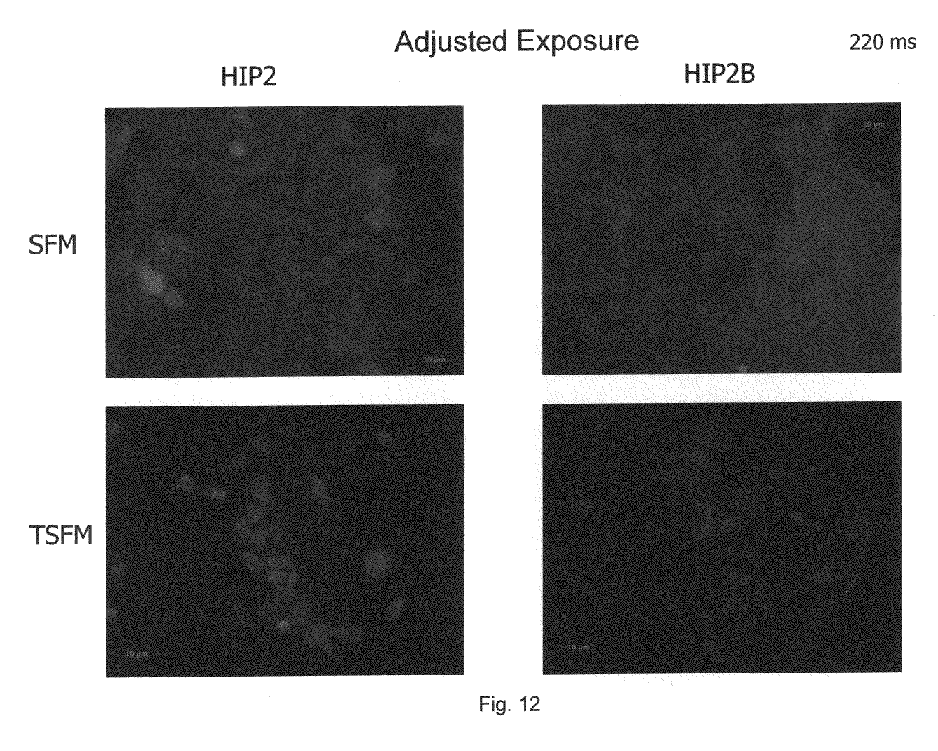

FIG. 12 demonstrates rabbit anti-human HIP receptor antibody labeled with Cy3 in (A) TSFM alone and (B) TSFM with 150 μM (HIP) for 48 hours. HIP stimulates the receptor membrane bound protein to be engulfed by the cytoplasmic membrane and transported to the nuclear membrane.

Although the present invention has been described in considerable detail with reference to certain preferred embodiments thereof other versions are possible. Therefore the spirit and scope of the appended claims should not be limited to the description and the preferred versions contained within this specification.

1. A method of treating newly diagnosed or preexisting type 1 diabetes comprising administering an islet neogenesis agent selected from hamster INGAP (SEQ ID No. 1), HIP3 (SEQ ID No. 2), HIP1 (SEQ ID No. 3), HIP2 (SEQ ID No. 4), HIP3B (SEQ ID No. 5), HIP1B (SEQ ID No. 6), HIP2B (SEQ ID No. 7), HIP3Cys (SEQ ID No. 8), HIP1Cys (SEQ ID No. 9). HIP2Cys (SEQ ID No. 10), HIP3CysDimer (SEQ ID No. 11), HIP1CysDimer (SEQ ID No. 12), HIP2CysDimer (SEQ ID No. 13), HIP3CysBlocked (SEQ ID No. 14), HIP1CysBlocked (SEQ ID No. 15), HIP2CysBlocked (SEQ ID No. 16), HIP3CysBlockedDimer (SEQ ID No. 17), HIP1CysBlockedDimer (SEQ ID No. 18), HIP2CysBlockedDimer (SEQ ID No. 19), HIP3CysPEG (SEQ ID No. 20), HIP1Cys PEG (SEQ ID No. 21), HIP2CysPEG (SEQ ID No. 22), HIP3CysBlockedPEG (SEQ ID No. 23), HIP1CysBlockedPEG (SEQ ID No. 24), and HIP2CysBlockedPEG (SEQ ID No. 25) in combination with an immune tolerance, agent selected from mycophenolate mofetil, daclizumab, rituximab, an anti CD3 antibody, abatacept, alemtuzumab, a humanized monoclonal antibody to T-cells, polyclonal anti-T-lymphocyte globulin (ATG), DiaPep277, anti-GAD antibody vaccine based on the 65 kDa isoform of the recombinant human glutamic acid decarboxylase protein, and diazoxide.

2. The method of claim 1, wherein said anti CD3 antibody is selected from teplizumab and TRX4.

3. The method of claim 1, wherein said islet neogenesis agent is selected from HIP3 (SEQ ID No. 2), HIP1 (SEQ ID No. 3), HIP2 (SEQ ID No. 4), HIP3B (SEQ ID No. 5), HIP1B (SEQ ID No. 6). HIP2B (SEQ ID No. 7), HIP3Cys (SEQ ID No. 8), HIP1Cys (SEQ ID No. 9), HIP2Cys (SEQ ID No. 10), HIP3CysDimer (SEQ ID No. 11), HIP1CysDimer (SEQ ID No. 12), HIP2CysDimer (SEQ ID No. 13), HIP3CysBlocked (SEQ ID No. 14), HIP1CysBlocked (SEQ ID No. 15), HIP2CysBlocked (SEQ ID No. 16), HIP3CysBlockedDimer (SEQ ID No. 17), HIP1CysBlockedDimer (SEQ ID No. 18), HIP2CysBlockedDimer (SEQ ID No. 19), HIP3CysPEG (SEQ ID No. 20), HIP1CysPEG (SEQ ID No. 21), HIP2CysPEG (SEQ ID No. 22), HIP3CysBlockedPEG (SEQ ID No. 23), HIP1CysBlockedPEG (SEQ ID No. 24), and HIP2CysBlockedPEG (SEQ ID No. 25).

4. The method of claim 1, wherein said islet neogenesis agent is selected from HIP2 (SEQ ID No. 4) and HIP2B (SEQ ID No. 7).

5. The method of claim 1, wherein said islet neogenesis agent is administered in a therapeutically effective amount.

6. The method of claim 5, wherein the therapeutically effective amount is from about 5 to about 150 mg/kg/day.

7. The method of claim 1, wherein said immune tolerance agent is administered in a therapeutically effective amount.

8. The method of claim 1 further comprising administering vitamin D or derivatives thereof.

9. The method of claim 8, wherein said vitamin D or derivative thereof is selected from cholecalciferol and 1,25 dihydroxyvitamin D.

10. The method of claim 1 further comprising administering a beta cell or islet function optimizing agent selected from GLP-1 and its analogs, GIF, amylin pramlintide. GLP-1 receptor agonists, exendin-4/exenatide, DPP-4 inhibitors, gastrin, epidermal growth, factor-1, thiazolidinedione, AGI-1067, CB1 receptor antagonists, gut peptide, peptide YY, neuropeptide Y, drugs that impact the leptin, ghrelin, pro-opiomelanocortin/melanocortin pathways or the melanocortin receptor, orlistat, sibutramine and acarbose.

11. The method of claim 10, wherein said DPP-4 inhibitor is selected from vildagliptin, sitagliptin, saxagliptin, and PHX1149.

12. The method of claim 10, wherein said thiazolidinedione is selected from rosiglitazone and pioglitazone.

13. The method of claim 10, wherein said CB1 receptor antagonist is rimonabant.

14. The method of claim 10, wherein said GLP-1 receptor agonist is liraglutide.

15. A method of treating newly diagnosed or preexisting type 2 diabetes comprising administering an islet neogenesis agent selected from hamster INGAP (SEQ ID No. 1), HIP3 (SEQ ID No. 2), HIP1 (SEQ ID No. 3), HIP2 (SEQ ID No. 4), HIP3B (SEQ ID No. 5), HIP1B (SEQ ID No. 6), HIP2B (SEQ ID No. 7), HIP3Cys (SEQ ID No. 8), HIP1Cys (SEQ ID No. 9), HIP2Cys (SEQ ID No. 10), HIP3CysDimer (SEQ ID No. 11), HIP1CysDimer (SEQ ID No. 12), HIP2CysDimer (SEQ ID No. 13), HIP3CysBlocked (SEQ ID No. 14), HIP1CysBlocked (SEQ ID No. 15), HIP2CysBlocked (SEQ ID No. 16), HIP3CysBlockedDimer (SEQ ID No. 17), HIP1CysBlockedDimer (SEQ ID No. 18), HIP2CysBlockedDimer (SEQ ID No. 19), HIP3CysPEG (SEQ ID No. 20), HIP1CysPEG (SEQ ID No. 21), HIP2CysPEG (SEQ ID No. 22), HIP3CysBlockedPEG (SEQ ID No. 23), HIP1CysBlockedPEG (SEQ ID No. 24), and HIP2CysBlockedPEG (SEQ ID No. 25) in combination with a beta cell or islet function optimizing agent selected from GLP-1 and its analogs, GIP, amylin, pramlintide, GLP-1 receptor agonists, exendin-4/exenatide, DPP-4 inhibitors, gastrin, epidermal growth factor-1, metformin, thiazolidinedione, AGI-1067, CB1 receptor antagonists, gut peptide, peptide YY, neuropeptide Y, drags that impact the leptin, ghrelin, pro-opiomelanocortin/melanocortin pathways or the melanocortin receptor, orlistat, sibutramine and acarbose.

16. The method of claim 15, wherein said islet neogenesis agent is selected from HIP3 (SEQ ID No. 2), HIP1 (SEQ ID No. 3), HIP2 (SEQ ID No. 4), HIP3B (SEQ ID No. 5), HIP1B (SEQ ID No. 6), HIP2B (SEQ ID No. 7), HIP3Cys (SEQ ID No. 8), HIP1Cys (SEQ ID No. 9), HIP2Cys (SEQ ID No. 10), HIP3CysDimer (SEQ ID No. 11), HIP1CysDimer (SEQ ID No. 12), HIP2CysDimer (SEQ ID No. 13), HIP3CysBlocked (SEQ ID No. 14), HIP1CysBlocked (SEQ ID No. 15), HIP2CysBlocked (SEQ ID No. 16), HIP3CysBlockedDimer (SEQ ID No. 17), HIP1CysBlockedDimer (SEQ ID No. 18), HIP2CysBlockedDimer (SEQ ID No. 19), HIP3CysPEG (SEQ ID No. 20), HIP1CysPEG (SEQ ID No. 21), HIP2CysPEG (SEQ ID No. 22), HIP3CysBlockedPEG (SEQ ID No. 23), HIP1CysBlockedPEG (SEQ ID No. 24), and HIP2CysBlockedPEG (SEQ ID No. 25).

17. The method of claim 15, wherein said islet neogenesis agent is selected from HIP2 (SEQ ID No. 4) and HIP2B (SEQ ID No. 7).

18. The method of claim 15, wherein said DPP-4 inhibitor is selected from vildagliptin, sitagliptin, saxagliptin, and PHX1149.

19. The method of claim 15, wherein said thiazolidinedione is selected from rosiglitazone and pioglitazone.

20. The method of claim 15, wherein said CB1 receptor antagonist is rimonabant.

21. The method of claim 15, wherein said GLP-1 receptor agonist is liraglutide.

22. The method of claim 15, wherein said islet neogenesis agent is administered in a therapeutically effective amount.

23. The method of claim 22, wherein the therapeutically effective amount is from about 5 to about 150 mg/kg/day.

24. The method of claim 15, wherein said beta cell or islet function optimizing agent is administered in a therapeutically effective amount.

25. The method of claim 15 further comprising administering vitamin D or derivative thereof.

26. The method of claim 25, wherein said vitamin D or derivative thereof is selected from cholecalciferol and 1,25 dihydroxyvitamin D.

27. A method of treating a pathology associated with impaired pancreatic function comprising administering an islet neogenesis agent selected from hamster INGAP (SEQ ID No. 1), HIP3 (SEQ ID No. 2), HIP1 (SEQ ID No. 3), HIP2 (SEQ ID No. 4), HIP3B (SEQ ID No. 5), HIP1B (SEQ ID No. 6), HIP2B (SEQ ID No. 7), HIP3Cys (SEQ ID No. 8), HIP1Cys (SEQ ID No. 9). HIP2Cys (SEQ ID No. 10), HIP3CysDimer (SEQ ID No. 11), HIP1CysDimer (SEQ ID No. 12), HIP2CysDimer (SEQ ID No. 13), HIP3CysBlocked (SEQ ID No. 14), HIP1CysBlocked (SEQ ID No. 15), HIP2CysBlocked (SEQ ID No. 16). HIP1CysBlockedDimer (SEQ ID No. 17), HIP1CysBlockedDimer (SEQ ID No. 18), HIP2CysBlockedDimer (SEQ ID No. 19), HIP3CysPEG (SEQ ID No. 20), HIP1CysPEG (SEQ ID No. 21), HIP2CysPEG (SEQ ID No. 22), HIP3CysBlockedPEG (SEQ ID No. 23), HIP1CysBlockedPEG (SEQ ID No. 24), and HIP2CysBlockedPEG (SEQ ID No. 25) in combination with a beta cell or islet function optimizing agent selected from GUM and its analogs, GIF, amylin, pramlintide, GLP-1 receptor agonists, exendin-4/exenatide, DPP-4 inhibitors, gastrin, epidermal growth factor-1, metformin, thiazolidinedione, AGI-1067, CB1 receptor antagonists, gut peptide, peptide YY, neuropeptide Y, drugs that impact the leptin, ghrelin, pro-opiomelanocortin/melanocortin pathways or the melanocortin receptor, orlistat, sibutramine, and acarbose.

28. The method of claim 27, wherein said pathology associated with impaired pancreatic function is selected from latent autoimmune diabetes of adulthood, pre-diabetes, impaired fasting glucose, impaired glucose tolerance, fasting hyperglycemia, insulin resistant syndrome, hyperglycemic conditions, abnormal fasting glucose or insulin levels, metabolic syndrome, overweight, obesity, hyperlipidemia, cholesterolemia, hypertriglyceridemia, eating disorders, polycystic ovarian syndrome, anovulatory cycles, fasting hyperlipidemia, fasting hypercholesterolemia, elevated fasting total cholesterol, elevated LDL and VLDL cholesterol, family history of diabetes and tonus of impotence or sexual dysfunction associated with such conditions.

29. The method of claim 27, wherein said islet neogenesis agent is selected from HIP3 (SEQ ID No. 2), HIP1 (SEQ ID No. 3), HIP2 (SEQ ID No. 4), HIP3B (SEQ ID No. 5), HIP1B (SEQ ID No. 6), HIP2B (SEQ ID No. 7), HIP3Cys (SEQ ID No. 8), HIP1Cys (SEQ ID No. 9), HIP2Cys (SEQ ID No. 10), HIP3CysDimer (SEQ ID No. 11), HIP1CysDimer (SEQ ID No. 12), HIP2CysDimer (SEQ ID No. 13), HIP 3 CysBlocked (SEQ ID No. 14), HIP1CysBlocked (SEQ ID No. 15), HIP2CysBlocked (SEQ ID No. 16), HIP3CysBlockedDimer (SEQ ID No. 17), HIP1CysBlockedDimer (SEQ ID No. 18), HIP2CysBlockedDimer (SEQ ID No. 19), HIP3CysPEG (SEQ ID No. 20), HIP1CysPEG (SEQ ID No. 21), HIP2CysPEG (SEQ ID No. 22), HIP3CysBlockedPEG (SEQ ID No. 23), HIP1CysBlockedPEG (SEQ ID No. 24), and HIP2CysBlockedPEG (SEQ ID No. 25).

30. The method of claim 27, wherein said islet-neogenesis agent is selected from HIP2 (SEQ ID No. 4) and HIP2B (SEQ ID No. 7).

31. The method of claim 27, wherein said DPP-4 inhibitor is selected from vildagliptin, sitagliptin, saxagliptin, and PHX1149.

32. The method of claim 27, wherein said thiazolidinedione is selected from rosiglitazone and pioglitazone.

33. The method of claim 27, wherein said CB1 receptor antagonist is rimonabant.

34. The method of claim 27, wherein said GLP-1 receptor agonist is liraglutide.

35. The method of claim 27, wherein said islet neogenesis agent is administered in a therapeutically effective amount.

36. The method of claim 37, wherein the therapeutically effective-amount is from about 5 to about 150 mg/kg/day.

37. The method of claim 27, wherein said beta cell or islet function optimizing agent is administered in a therapeutically effective amount.

38. The method of claim 27 further comprising administering vitamin D or derivative thereof.

39. The method of claim 38, wherein said vitamin D or derivative thereof is selected from cholecalciferol and 1,25 dihydroxyvitamin D.
